(19) United States
(12) Patent Application Publication
(10) Pub. No.: US 2024/0009236 A1
(43) Pub. Date: Jan. 11, 2024

(54) MODIFIED NK CELLS WITH REDUCED CCR5 EXPRESSION AND METHODS OF THEIR USE

(71) Applicant: The United States of America, as represented by the Secretary, Dept. of Health and Human Services, Bethesda, MD (US)

(72) Inventors: Richard W. Childs, Bethesda, MD (US); Emily R. Levy, Bethesda, MD (US); Joseph Clara, Bethesda, MD (US)

(73) Assignee: The United States of America, as represented by the Secretary, Dept. of Health and Human Services, Bethesda, MD (US)

(21) Appl. No.: 18/035,240

(22) PCT Filed: Nov. 3, 2021
(86) PCT No.: PCT/US2021/057845

§ 371 Date: May 3, 2023

Related U.S. Application Data
(60) Provisional application No. 63/109,707, filed on Nov. 4, 2020.

(51) Int. Cl. A61K 35/17; C12N 5/0783; A61P 35/00

(52) U.S. Cl. CPC A61K 35/17; C12N 5/0646; A61P 35/00

(57) ABSTRACT

Modified NK cells with reduced expression of CCR5 are provided. Methods of treating a subject with cancer with the modified NK cells are also provided. In some examples, the modified NK cells also have reduced expression of one or more of CCR1, CXCR6, and CD38, increased expression of one or more of CXCR4, CCR7, and CXCR3, and/or express a chimeric antigen receptor.

### CROSS REFERENCE TO RELATED APPLICATION

This application claims the benefit of U.S. Provisional Application No. 63/109,707, filed Nov. 4, 2020, which is incorporate herein by reference in its entirety.

### FIELD OF THE DISCLOSURE

Modified NK cells with reduced C-C chemokine receptor 5 (CCR5) expression and methods of their use, for example for treating cancer, are disclosed.

### BACKGROUND

Allogeneic and autologous natural killer (NK) cell-based therapies have proven to be safe in patients with hematological malignancies across multiple clinical trials. However, at present, only a small percentage of patients who have received NK cell based therapy have achieved complete remissions. NK cells must first traffic into the tumor microenvironment in order to effectively lyse tumor targets in vivo. Many hematological malignancies reside in or metastasize through bone marrow (BM) compartments. Recent studies in both murine and macaque models evaluating NK cell distribution following intravenous (i.v.) infusion unexpectedly revealed preferential trafficking into liver tissue over BM niches, which for hematological malignancies is undesirable. Retention in the liver is also undesirable for treatment of solid tumors.

### SUMMARY OF THE DISCLOSURE

Disclosed herein are modified NK cells with decreased targeting to or retention in the liver, or both. In some embodiments, the NK cells also include modifications that increase targeting to or retention in a tissue or compartment of interest, such as the blood, bone marrow, lymph node, or tumor microenvironment.

In some embodiments, modified ex vivo expanded natural killer (NK) cells having reduced expression of C-C chemokine receptor 5 (CCR5) compared to unmodified ex vivo expanded NK cells are provided. In some examples, the modified NK cells include a deletion of at least one nucleotide and/or an insertion of at least one nucleotide in genomic DNA encoding CCR5, for example a deletion of at least one or more nucleotides and/or an insertion of one or more nucleotides in exon 2 of CCR5. The insertion and/or deletion of nucleotides in some examples may be produced by CRISPR/Cas9 gene editing. In other examples, the modified NK cell has a reduced amount of CCR5-encoding mRNA, which in some examples may be produced by siRNA gene silencing.

In some embodiments, the modified NK cells have reduced expression of CCR5 and also reduced expression of one or more additional genes, increased expression of one or more additional genes, expression of a chimeric antigen receptor, or a combination of any two or more thereof. In some examples, the modified NK cell with reduced expression of CCR5 also has increased expression of any one of C-X-C chemokine receptor 4 (CXCR4), C-C chemokine receptor 7 (CCR7), and/or a C-X-C chemokine receptor 3 (CXCR3) compared to an unmodified ex vivo expanded NK cell. In some examples, the modified NK cells include a heterologous nucleic acid encoding one or more of CXCR4, CCR7, or CXCR3. In other examples, the modified NK cell with reduced expression of CCR5 also includes reduced expression of any one of C-C chemokine receptor 1 (CCR1), C-X-C chemokine receptor 6 (CXCR6), and/or CD38 compared to an unmodified ex vivo expanded NK cell. In further examples, the modified NK cell with reduced expression of CCR5 also expresses a chimeric antigen receptor (for example, includes a heterologous nucleic acid encoding a chimeric antigen receptor).

Also provided in some embodiments is a composition including a modified NK cell disclosed herein and a pharmaceutically acceptable carrier.

In additional embodiments, methods of treating a subject with cancer are provided. The methods include administering an effective amount of modified NK cells with reduced expression of CCR5 (and optionally with reduced expression of one or more additional genes, increased expression of one or more genes, and/or expressing a chimeric antigen receptor) to the subject. In particular examples, the NK cells are autologous to the subject. The subject may have a solid tumor or a hematological malignancy (for example, multiple myeloma). In additional examples, the methods also include administering to the subject an additional therapy, such as an anti-cancer antibody.

Methods of producing the disclosed modified NK cells are also provided. In some examples, the methods include expanding a population of isolated NK cells in a cell culture medium comprising interleukin-2 and/or interleukin-15 for 2-5 days to produce a population of expanded NK cells, making a modification to reduce expression of CCR5 in the population of expanded NK cells (e.g., introducing a ribonucleoprotein comprising at least one sgRNA targeting CCR5 and Cas9) to produce a population of modified NK cells, and culturing (e.g., further expanding) the population of modified NK cells in a cell culture medium comprising interleukin-2 and/or interleukin-15 for 3-15 days to produce the modified NK cells. In some examples, the NK cells are cultured in the presence of irradiated feeder cells (such as an Epstein-Barr virus transformed lymphoblastoid cell line or a genetically modified K562 cell line). If feeder cells are used, the methods may also include separating the feeder cells and the modified NK cells. In some non-limiting examples, the expanded NK cells are contacted with at least one sgRNA (e.g., 1, 2, or 3 sgRNAs) having the nucleic acid sequence of any one of SEQ ID NOs: 1-3.

In some embodiments, the methods further include making an additional modification to decrease expression of one or more additional genes, for example, introducing into the expanded NK cells a ribonucleoprotein including at least one sgRNA targeting CCR1, at least one sgRNA targeting CXCR6, and/or at least one sgRNA targeting CD38, and Cas9. In other embodiments, the expanded NK cells are transduced with a viral vector comprising one or more heterologous nucleic acids encoding one or more of CXCR3, CXCR4, CCR7, and a chimeric antigen receptor.

The foregoing and other features and advantages of the disclosure will become more apparent from the following detailed description of several embodiments which proceeds with reference to the accompanying figures.

### BRIEF DESCRIPTION OF THE FIGURES

FIGS. 1A and 1B show analysis of NK cell transcriptional changes after ex vivo expansion with feeder cells. FIG. 1A is a schematic of the experimental protocol for analyzing transcriptional and phenotypic changes in NK cells due to ex vivo expansion with feeder cells. FIG. 1B shows the frequency of viable CD56⁺CD3⁻ cells and CD3 contamination within the samples that were utilized for phenotype and RNA-seq experiments. Fresh (F) NK cells were isolated from healthy donor buffy coats (n=6) and then expanded with either EBV-LCL (L) or GE-K562 (K) feeder cells.

FIGS. 2A-2E illustrate that the NK cell transcriptional landscape is heavily altered due to ex vivo expansion with feeder cells. F=freshly isolated and rested NK cells, L=LCL-expanded NK cells, and K=GE-K562-expanded NK cells. FIG. 2A shows principal component analysis of fresh and expanded NK cells. FIG. 2B shows unsupervised gene clustering of fresh and expanded NK cell populations. FIG. 2C shows volcano plots to illustrate the distribution of significance [−log₁₀(adjusted p value)] and change in expression [log 2 (fold change)] of differentially expressed genes within the comparisons. FIG. 2D shows gene ontology enrichment of top significant gene groups that are differentially expressed. FIG. 2E shows a Scree plot for PCA of the RNA-sequenced NK cell populations and a heat map that represents differential expression of significant DEGs between LCL and GE-K562 expanded NK cells (FPKM).

FIGS. 3A and 3B show that ex vivo expansion of NK cells leads to highly significant transcriptional alterations in genes that are important for NK cell cytotoxicity. FIG. 3A is a bubble plot that displays genes that control NK cell cytotoxicity. Axis values represent log₂-transformed fold change of transcript expression in expanded NK cells compared to fresh NK cells, where L=LCL−expanded NK cells and K=GE-K562-expanded NK cells. The size of the bubble corresponds to the log₁₀-transformed adjusted p value to denote the statistical significance of differential expression, based on LCL-expanded NK cell values. FIG. 3B shows heat maps that display FPKM values of the genes that are graphed in FIG. 3A.

FIGS. 4A-4C illustrate that ex vivo expansion of NK cells leads to highly significant transcriptional alterations in genes that govern cellular trafficking. FIG. 4A shows bubble plots that display genes that control NK in vivo trafficking. Graphical organization is the same as in FIG. 3A. FIG. 4B shows heat maps that report FPKM values for the genes that are graphed in FIG. 4A. FIG. 4C is a series of line graphs showing the change in gene transcription in NK cells from fresh (F) to expanded with either LCL feeder cells (L) or GE-K562 feeder cells (K), from individual donors. The paired T-test was used to determine significance, ns=not significant, *p<0.05, **p<0.01, ***p<0.001.

FIG. 5 shows a bubble plot of genes that are included in Table 3. Graphical organization is the same as in FIG. 3A. The genes that are labeled have a log 2(fold change) value of >3 or <−3. Significance is reported as −log₁₀(adjusted p value).

FIGS. 6A and 6B show that ex vivo expansion with feeder cells induces a chemotactic receptor and adhesion molecule expression shift on NK cells. FIG. 6A shows surface expression phenotype on expanded NK cells, compared to non-expanded NK cells. Data are reported as fold change [(final-initial)/initial] of geometric mean florescent intensities relative to FMO controls (rGMFI). Black bars represent surface expression on NK cells that were expanded with EBV-LCL feeder cells and white bars represent surface expression on NK cells that were expanded with GE-K562 feeder cells. Error bars report the SEM and significance was analyzed with a paired T-test (n=6 donors). FIG. 6B shows histograms that represent surface expression levels of chemotactic receptors.

FIGS. 7A and 7B illustrate that ex vivo expansion of NK cells with SMI-EBV-LCL feeder cells creates a concentrated and chronic pro-inflammatory environment. FIG. 7A shows plots from NK cell culture supernatants analyzed using a multiplex assay for the presence of various cytokines and chemokines. Molecule concentrations are reported as pg/mL over time. Line graphs show data from supernatants collected from NK cell expansion with EBV-LCL feeder cells or from supernatants of NK cell alone in IL-2 media (n=2 technical replicates, 2 biological replicates). FIG. 7B shows surface expression changes of chemotactic receptors in response to exposure to individual various cytokines and chemokines for 24 hours in vitro (n=2).

FIGS. 8A-8C show that reduction of CCR5 expression significantly reduces NK cell trafficking into liver 24 hours following infusion of modified NK cells into mice. FIG. 8A shows bar graphs that illustrate CCR5 positivity and surface expression on ex vivo expanded NK cells (n=4 donors). FIG. 8B shows dot plots representing CCR5 surface expression on NK cells (one representative donor). FIG. 8C shows plots displaying the fraction of NK cells that were recovered in tissues, 24 hours following i.v. infusion into mice (n=8-9 per group, two independent experiments). Statistics determined with the Students T-test, two tailed, ns=not significant, *p<0.05, **p<0.01, ****p<0.001.

FIG. 9 is a schematic diagram of an exemplary clinical protocol using CCR5 knockout NK cells for treating a patient with cancer.

### SEQUENCE LISTING

Any nucleic acid and amino acid sequences listed herein or in the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NOs: 1-3 are synthetic guide RNAs for CRISPR/Cas9 KO of CCR5.

### DETAILED DESCRIPTION

Ex vivo NK cell expansion using feeder cells is commonly used in clinical trials to grow and activate allogenic and autologous NK cell products prior to infusion into patients. Here, it is disclosed that the transcriptional landscape of NK cells changes dramatically by this process. Remarkably, ex vivo expanded NK cells have substantial alterations in expression of a number of genes that impact in vivo cellular trafficking including CCR5, CCR1, CXCR3, and CXCR4. This shift in chemokine receptor gene expression appears to compromise NK cell homing to the bone marrow, for example by promoting NK cell homing to and retention in the liver. As disclosed herein, gene editing (such as CRISPR gene editing) to reduce expression of one of these genes (CCR5) can be utilized redirect the homing of ex vivo expanded NK cells from the liver into the circulation following infusion into immunodeficient mice. This work therefore provides opportunities to bolster the effectiveness of NK cell immunotherapy for hematological malignancies and other cancers.

### I. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Redei, Encyclopedic Dictionary of Genetics, Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety. All GenBank Accession Nos. are incorporated as present in GenBank on Nov. 4, 2020. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Cancer: Also referred to herein as a “malignant tumor” or “malignant neoplasm.” Any of a number of diseases that are characterized by uncontrolled, abnormal proliferation of cells, the potential of cancer cells to spread locally or through the bloodstream and lymphatic system to other parts of the body (e.g., metastasize), as well as any of a number of characteristic structural and/or molecular features. A “cancer cell” is a cell having specific structural properties, lacking differentiation, and being capable of invasion and metastasis. As used herein, cancer refers to both solid tumors and hematological malignancies.

C-C motif chemokine receptor 1 (CCR1): A member of the beta chemokine receptor family, whose ligands include macrophage inflammatory protein 1 α (MIP-1α), regulated on activation normal T expressed and secreted proteins (RANTES), monocyte chemoattractant protein 3 (MCP-3), and myeloid progenitor inhibitory factor-1 (MPIF-1). Exemplary human CCR1 sequences include GenBank Accession Nos. NM 001295 (nucleic acid sequence) and NP 001286 (amino acid sequence), and exemplary mouse CCR1 sequences include GenBank Accession Nos. NM 009912 (nucleic acid sequence) and NP 034042 (amino acid sequence), all of which are incorporated herein by reference.

C-C motif chemokine receptor 5 (CCR5): A member of the beta chemokine receptor family, whose ligands include monocyte chemoattractant protein-2 (MCP-2), macrophage inflammatory protein 1 α (MIP-1α), macrophage inflammatory protein 1β (MIP-1β), and regulated on activation normal T expressed and secreted proteins (RANTES). In at least some examples, CCR5 signaling plays a role in targeting cells (such as NK cells) to the liver. Exemplary human CCR5 sequences include GenBank Accession Nos. NM 000579 and NM 001100168 (nucleic acid sequences) and NP 000570 and NP 001093638 (amino acid sequences), and exemplary mouse CCR5 sequences include GenBank Accession Nos. NM 009917 (nucleic acid sequence) and NP 034047 (amino acid sequence), all of which are incorporated herein by reference.

C-C motif chemokine receptor 7 (CCR7): A member of the chemokine receptor family that binds to chemokine ligand 19 (CCL19). The CCR7 signaling pathway appears to increase targeting of cells (such as NK cells) to bone marrow or lymph nodes. Exemplary human CCR7 sequences include GenBank Accession Nos. NM_001301714, NM_001838, NM_001301717, NM_001301716, and NM_001301718 (nucleic acid sequences) and NP_001288643, NP_001829, NP_001288646, NP_001288645, and NP_001288647 (amino acid sequences), and exemplary mouse CCR7 sequences include GenBank Accession Nos. NM_001301713 and NM_007719 (nucleic acid sequences) and NP_001288642 and NP_031745 (amino acid sequences), all of which are incorporated herein by reference.

C-X-C motif chemokine receptor 3 (CXCR3): A member of the chemokine receptor family whose ligands include monokine induced by interferon-γ (CXCL9/Mig), interferon-γ inducible 10 kDa protein (CXCL10/IP10), interferon inducible T cell a-chemoattractant (CXCL11/I-TAC), and CXCL4/PF4. Signaling by CXCR3 is involved in cell trafficking to sites of inflammation and to solid tumor microenvironment. Exemplary human CXCR3 sequences include GenBank Accession Nos. NM_001504 and NM_001142797 (nucleic acid sequences) and NP_001495 and NP_001136269 (amino acid sequences), and exemplary mouse CXCR3 sequences include GenBank Accession Nos. NM_009910 (nucleic acid sequence) and NP_034040 (amino acid sequence), all of which are incorporated herein by reference.

C-X-C motif chemokine receptor 4 (CXCR4): A member of the CXC chemokine receptor family that binds to stromal cell-derived factor 1 (SDF1). The CXCR3 signaling pathway appears to increase targeting of cells (such as NK cells) to bone marrow or lymph nodes. Exemplary human CXCR4 sequences include GenBank Accession Nos. NM_003467, NM_001348059, NM_001348056, NM_001348060, and NM_001008540 (nucleic acid sequences) and NP_003458, NP_001334988, NP_001334985, NP_001334989, and NP_001008540 (amino acid sequences), and exemplary mouse CXCR4 sequences include GenBank Accession Nos. NM_001356509 and NM_009911 (nucleic acid sequences) and NP_001343438, and NP_034041 (amino acid sequences), all of which are incorporated herein by reference.

C-X-C motif chemokine receptor 6 (CXCR6): A member of the CXC chemokine receptor family that binds to chemokine ligand 16 (CCL16). The CXCR6 signaling pathway regulates cell migration to various tissues, including liver, spleen, and lungs. Exemplary human CXCR6 sequences include GenBank Accession Nos. NM_006564 (nucleic acid sequence) and NP_006555 (amino acid sequence), and exemplary mouse CXCR6 sequences include GenBank Accession Nos. NM_030712 (nucleic acid sequence) and NP_109637 (amino acid sequence), all of which are incorporated herein by reference.

CD38: The CD38 molecule is a type II transmembrane glycoprotein with a molecular weight of approximately 45 kDa. It is a bifunctional ectoenzyme, capable of catalyzing conversion of nicotinamide adenine dinucleotide (NAD+) to cyclic ADP-ribose (cADPR) and conversion of cADPR into ADP-ribose. CD38 is also involved in cell adhesion and cellular signaling, including calcium mobilization. Exemplary human CD38 sequences include GenBank Accession Nos. NM_001775 (nucleic acid sequence) and NP_001766 (amino acid sequence), both of which are incorporated herein by reference.

Culturing or Cell culture: Growth of a population of cells in a defined set of conditions (such as culture medium, extracellular matrix, temperature, and/or time of culture) in vitro or ex vivo. In some examples, a cell culture includes a substantially pure culture (for example, isolated NK cells). In additional examples a cell culture includes a mixed culture, such as co-culture of two or more types of cells (for example a culture of NK cells with feeder cells). In further examples, a cell culture includes cells grown in contact with an extracellular matrix.

Culture Medium: A synthetic set of culture conditions with the nutrients necessary to support the viability, function, and/or growth of a specific population of cells, such as NK cells. Culture media generally include components such as a carbon source, a nitrogen source and a buffer to maintain pH. Additional components in culture media also may include one or more of serum (such as heat-inactivated serum), cytokines, hormones, growth factors, protease inhibitors, protein hydrolysates, shear force protectors, proteins, vitamins, glutamine, trace elements, inorganic salts, minerals, lipids, and/or attachment factors.

Cytokine: Proteins made by cells that affect the behavior of other cells, such as lymphocytes. In one embodiment, a cytokine is a chemokine, a molecule that affects cellular trafficking. The term “cytokine” is used as a generic name for a diverse group of soluble proteins and peptides that act as humoral regulators at nanomolar to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to, tumor necrosis factor α (TNF-α), interleukin-2 (IL-2), interleukin-7 (IL-7), interleukin-15 (IL-15), interleukin-21 (IL-21), and interferon-γ (IFN-γ).

Effective amount: A quantity of a specified agent sufficient to achieve a desired effect, for example, in a subject being treated with that agent. In some examples, an effective amount of the modified NK cells disclosed herein is an amount sufficient to treat or inhibit a disease or disorder in a subject (such as a solid tumor or hematological malignancy). In other examples, an effective amount is an amount of modified NK cells sufficient to reduce or ameliorate one or more symptoms of a disease or disorder in a subject. The effective amount (for example an amount ameliorating, inhibiting, and/or treating a disorder in a subject) will be dependent on, for example, the particular disorder being treated, the subject being treated, the manner of administration of the composition, and other factors.

Expression: The process by which the coded information of a gene is converted into an operational, non-operational, or structural part of a cell, such as the synthesis of a protein. Gene expression can be influenced by external signals (such as a hormone). Expression of a gene also can be regulated anywhere in the pathway from DNA to RNA to protein. Regulation can include controls on or regulation of transcription, translation, RNA transport and processing, degradation of intermediary molecules such as mRNA, or through activation, inactivation, compartmentalization or degradation of specific protein molecules after they are produced.

Alterations in the expression of a nucleic acid molecule can be associated with, and in fact cause, a change in expression (such as amount and/or activity) of the corresponding protein. Alterations in gene expression, include but are not limited to overexpression (e.g., increased amount and/or activity of the nucleic acid or protein) and underexpression (e.g., decreased amount and/or activity of the nucleic acid or protein). Controls or standards for comparison to a sample, for the determination of altered gene expression, include samples believed to be normal (in that they are not altered for the desired characteristic) as well as laboratory values, even though possibly arbitrarily set, keeping in mind that such values can vary from laboratory to laboratory. Laboratory standards and values may be set based on a known or determined population value and can be supplied in the format of a graph or table that permits comparison of measured, experimentally determined values.

Feeder cells: Cells that provide support for another cell type in ex vivo or in vitro culture. Feeder cells may provide one or more factors required for survival, growth, and/or differentiation (or inhibiting differentiation) of the cells cultured with the feeder cells. Typically feeder cells are irradiated or otherwise treated to prevent their proliferation in culture. In some examples disclosed herein, NK cells are cultured with feeder cells, such as irradiated EBV-transformed lymphoblast cells (e.g., EBV-LCL cells).

Isolated: An “isolated” or “purified” biological component (such as a cell, nucleic acid, peptide, or protein) has been substantially separated, produced apart from, or purified away from other components (for example, other biological components in the cell or the organism in which the component naturally occurs). Cells, nucleic acids, peptides and proteins that have been “isolated” or “purified” thus include cells, nucleic acids, and proteins purified by standard purification methods.

The term “isolated” or “purified” does not require absolute purity; rather, it is intended as a relative term. Thus, for example, an isolated biological component is one in which the biological component is more enriched than the biological component is in its natural environment within a cell, organism, sample, or production vessel (for example, a cell culture system). Preferably, a preparation is purified such that the biological component represents at least 50%, such as at least 70%, at least 80%, at least 90%, at least 95%, or greater, of the total biological component content of the preparation.

Natural Killer (NK) cells: Cells of the immune system that kill target cells in the absence of a specific antigenic stimulus and without restriction according to MHC class. In some examples, the target cells are cancer cells (such as solid tumor or hematological malignancy cells). NK cells are characterized by the presence of CD56 and the absence of CD3 surface markers. NK cells typically comprise approximately 10 to 15% of the mononuclear cell fraction in normal peripheral blood. Historically, NK cells were first identified by their ability to lyse certain tumor cells without prior immunization or activation. NK cells are thought to provide a “back up” protective mechanism against viruses and tumors that might escape the CTL response by down-regulating MHC class I presentation. In addition to being involved in direct cytotoxic killing, NK cells also serve a role in cytokine production, which can be important to control cancer and infection.

An expanded NK cell is refers to an NK cell that has been derived from a primary NK cell by ex vivo or in vitro cell culture in the presence of one or more cytokines (such as IL-2) for a period of time in order to increase the number of NK cells. Expanded NK cells exhibit altered gene expression compared to primary (non-expanded) NK cells, as described herein.

In some examples, a “modified NK cell” is a NK cell with increased and/or decreased expression of one or more genes compared to an unmodified NK cell. In some examples, a modified NK cell is transduced with a heterologous nucleic acid or expresses one or more heterologous proteins (in some examples, referred to as a “knockin”). In other examples, a modified NK cells has a modification that decreases expression of one or more genes (in some examples, referred to as a “knockout”). The terms “modified NK cell” and “transduced NK cell” are used interchangeably in some examples herein.

Pharmaceutically acceptable carrier: The pharmaceutically acceptable carriers (vehicles) useful in this disclosure are known to those of skill in the art. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compositions, such as one or more modified NK cells and/or additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Subject: A living multi-cellular vertebrate organism, a category that includes both humans and non-human mammals (such as mice, rats, dogs, cats, rabbits, sheep, horses, cows, and non-human primates).

Transduce: Transferring nucleic acid into a cell, such as transfer of a heterologous nucleic acid into a host cell. As used herein, the term transduce (or transfect or transform) includes all techniques by which a nucleic acid is introduced into a cell, including but not limited to transformation with plasmid vectors, infection with viral vectors or viral particles, and introduction of naked DNA by electroporation, nucleofection, lipofection, or particle gun acceleration.

Transgene: A heterologous nucleic acid introduced into a cell, for example, by transduction. In some examples, a transgene is a nucleic acid encoding a protein of interest. In other examples, a transgene includes a nucleic acid that is capable of modulating expression of a nucleic acid of interest, such as a sgRNA, small interfering RNA (siRNA), or antisense nucleic acid. The transgene may be operably linked to one or more expression control sequences, for example, a promoter.

A “heterologous” nucleic acid or protein refers to a nucleic acid or protein originating from a different genetic source. For example, a nucleic acid or protein that is heterologous to a cell originates from an organism or individual other than the cell in which it is expressed. In other examples, a heterologous nucleic acid or protein originates from a cell type other than the cell in which it is expressed (for example, a nucleic acid or protein not normally present in NK cells is heterologous to NK cells). In further examples, a heterologous nucleic acid includes a recombinant nucleic acid, such as a protein-encoding nucleic acid operably linked to a promoter from another gene and/or two or more operably linked nucleic acids from different sources.

Vector: A nucleic acid molecule allowing insertion of foreign or heterologous nucleic acid into a cell without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and/or translation of an inserted gene or genes. In some non-limiting examples, the vector is a viral vector, such as a retroviral vector or lentiviral vector.

### II. Modified NK Cells

Provided herein are modified NK cells with reduced expression of CCR5 compared to unmodified NK cells. This modification is useful to decrease targeting of the NK cells to the liver following ex vivo expansion and adoptive transfer to a subject. In some embodiments, the modified NK cells also have reduced expression of one or more additional genes and/or increased expression of one or more genes, compared to unmodified NK cells.

In some embodiments, the modified NK cells have reduced expression of CCR5 compared to an unmodified NK cell, such as compared to NK cells that have been expanded ex vivo. In some examples, the expression of CCR5 in a population of modified ex vivo expanded NK cells is reduced by at least about 25%, for example by at least about 50%, about 75%, about 85%, about 90%, about 95%, about 98%, about 99%, or more compared to unmodified ex vivo expanded NK cells. In some examples, the modified NK cells are referred to as “CCR5 knockout” (KO) cells, even though the cells may not have complete absence of CCR5 expression. For example, the modified expanded NK cells may have a reduction of CCR5 expression of at least about 50% (such as at least about 60%, at least about 70%, at least about 80%, at least about 90%, or at least about 95%) compared to unmodified expanded NK cells. In other examples, the number of CCR5 positive NK cells in a population of modified expanded NK cells may be reduced by at least about 50% (such as at least about 60%, at least about 70%, at least about 80%, at least about 90%, or at least about 95%) compared to the number of CCR5 positive cells in a population of unmodified expanded NK cells.

In some embodiments, CCR5 expression is reduced by disruption of the gene encoding CCR5, such as by a CRISPR/Cas9 disruption. Some exemplary methods of producing CCR5 KO NK cells are described in more detail in Section III, and Example 1. One of ordinary skill in the art can identify other methods of producing NK cells with reduced CCR5 expression.

In some embodiments, the modified NK cells have at least one disruption in the genomic DNA of the CCR5 gene, for example at least one nucleotide deletion or at least one nucleotide insertion (or both), resulting in decreased CCR5 expression (such as decreased amount of CCR5 mRNA or protein or decreased CCR5 protein activity). In one example, the disruption is in exon 2 of the CCR5 gene (e.g. in exon 2 as defined in GenBank Accession No. ENST00000292303). In particular examples, the modified NK cells have at least one disruption in exon 2 of the CCR5 gene introduced by CRISPR/Cas9 gene editing.

In additional embodiments, the modified NK cells have further modifications in addition to reduced expression of CCR5. These include modifications that result in any combination of one or more of reduced expression of CCR1, reduced expression of CXCR6, reduced expression of CD38, increased expression of CXCR4, increased expression of CCR7, increased expression of CXCR3, and expression of one or more chimeric antigen receptors (CARs). In some examples, cells with increased expression of an additional gene are referred to as having a “knockin” of the gene (e.g., “CCR7 knockin”). The CCR5 knockout reduces NK cell homing to the liver, while the additional knockouts or knockins increase homing to a tissue of interest (such as CXCR4 for homing to bone marrow or CCR7 for homing to lymph nodes) or provide other advantageous effects (such as CD38 knockout for increasing resistance of NK cells to anti-CD38 antibodies, or expression of therapeutic CARs).

In some examples, the expression of CCR1, CXCR6, and/or CD38 in the population of modified ex vivo expanded NK cells is reduced by at least about 25%, for example by at least about 50%, about 75%, about 85%, about 90%, about 95%, about 98%, about 99%, or more compared to unmodified ex vivo expanded NK cells. In other examples, the expression of CXCR4, CCR7, and/or CXCR3 is increased by at least about 25%, for example by at least about 50%, about 75%, about 90%, about 95%, about 100%, about 1.5-fold, about 2-fold, about 5-fold, or more, compared to unmodified ex vivo expanded NK cells.

In a particular example, the modified NK cells have reduced expression of CCR5 and increased expression of CXCR4. In another particular example, the modified NK cells have reduced expression of CCR5 and reduced expression of CXCR6, CCR1, or both. In other particular examples, the modified NK cells have reduced expression of CCR5 and increased expression of CCR7 or reduced expression of CCR5 and increased expression of CXCR3. In yet another example, the modified NK cells have reduced expression of CCR5, reduced expression of CD38, and increased expression of CXCR4. In a further particular example, the modified NK cells have reduced expression of CCR5 and expression of at least one CAR. Exemplary CARs that could be utilized include, but are not limited to a B cell maturation antigen (BCMA)-CAR, CD19-CAR, CD20-CAR, CD33-CAR, CD138-CAR, CS1-CAR, GD2-CAR, HER2-CAR, erbB2-CAR, carcinoembryonic antigen (CEA)-CAR, epithelial cell adhesion molecule (EpCAM)-CAR, natural-killer group 2, member D, long form (NKG2D-L)-CAR, or TRAIL receptor 1 (TRAIL-R1)-CAR.

### III. Methods of Producing Modified NK Cells

Disclosed herein are methods of producing modified NK cells (such as NK cells with reduced expression of one or more genes, for example, CCR5). In particular embodiments, the methods disclosed herein are utilized to reduce expression of one or more genes in ex vivo expanded NK cells. In additional embodiments, the methods also include increasing expression of one or more genes in ex vivo expanded NK cells, for example, utilizing transduction with one or more heterologous nucleic acids encoding protein(s) of interest. Exemplary methods for producing modified NK cells include (but are not limited to) those described in International Pat. Publ. No. WO 2019/089955, which is incorporated herein by reference in its entirety.

In some embodiments, the methods include expanding a population of isolated NK cells in a cell culture medium including interleukin-2, interleukin-15, or both for 2-5 days to produce a population of expanded NK cells, making a modification to reduce expression of CCR5 in the population of expanded NK cells to produce a population of modified expanded NK cells, and culturing the population of modified expanded NK cells in cell culture medium including interleukin-2, interleukin-15, or both for about 3-15 days. In some embodiments, the methods utilize a CRISPR/Cas9 system to reduce expression of a gene of interest, such as CCR5, in ex vivo expanded NK cells. Thus, in some examples, the methods include introducing a ribonucleoprotein (RNP) comprising at least one sgRNA targeting CCR5 and Cas9 into the population of expanded NK cells. In some examples, the RNP is introduced into the expanded NK cells by electroporation. Methods of CRISPR/Cas9 gene editing are known to one of ordinary skill in the art. In a particular example, the at least one sgRNA has the nucleic acid sequence of any one of SEQ ID NOs: 1-3. In some examples, sgRNAs include the nucleic acid sequence of each of SEQ ID NOs: 1-3 are introduced into the expanded NK cells. Other methods of reducing CCR5 expression include use of zinc finger nucleases or antisense nucleic acid techniques, such as siRNA silencing.

In some embodiments, the methods also include reducing expression of one or more additional genes in the ex vivo expanded NK cells, such as one or more of CCR1, CXCR6, and CD38. In other embodiments, the disclosed methods also include increasing expression of one or more genes in ex vivo expanded NK cells, such as those identified in Section II. In additional embodiments, the methods include reducing expression of one or more additional genes and increasing expression of one or more genes in ex vivo expanded NK cells. In some examples, the methods utilize a lentiviral system to introduce one or more heterologous nucleic acids (“transgenes”) encoding the gene(s) of interest into ex vivo expanded NK cells. In particular examples, expanded NK cells are transduced with a viral vector (such as a lentiviral vector) including one or more heterologous nucleic acids, for example, by incubating activated NK cells with the viral vector (for example, viral particles including the viral vector) for 1-3 days. In some examples, the one or more heterologous nucleic acids encode CCR7, CXCR4, CXCR3, and/or a CAR.

In particular embodiments, the NK cells are expanded (before and/or after modification) in the presence of feeder cells (such as irradiated feeder cells). Exemplary feeder cells include EBV-LCLs (e.g., TM-LCL, SMI-LCL), allogeneic or autologous PBMCs, Wilms tumor cell line HFWT, and K562 cells (such as genetically modified K562 cells, for example, K562-mb15-41BBL or K562-mbIL-21 cells). In some examples, NK cells are expanded in the presence of at least 1:1 ratio of feeder cells:NK cells, for example, at least 2:1, 5:1, 10:1, 15:1, 20:1, or more). The NK cells are also cultured with one or more cytokines (e.g., IL-2 and/or IL-15) throughout the process.

A. Isolation and Enrichment of NK Cells

Techniques for the in vitro isolation and enrichment of NK cells are described herein and are also known to one of ordinary skill in the art. Mononuclear cells are collected from a subject (such as a subject with cancer) or from a donor HLA-matched to the subject to be treated. In some examples, mononuclear cells are collected by an apheresis procedure. The mononuclear cells are enriched for NK cells, for example by negative depletion using an immuno-magnetic bead strategy. In some examples, NK cells are enriched by depleting the mononuclear cell sample of T cells, B cells, monocytes, dendritic cells, platelets, macrophages, and erythrocytes utilizing a mixture of biotinylated monoclonal antibodies. The non-NK cells in the sample are removed with magnetic beads coupled to streptavidin, resulting in an enriched preparation of NK cells. An exemplary commercially available kit for this method is Dynabeads® Untouched™ Human NK Cells kit (ThermoFisher Scientific, Waltham, MA). In another example, NK cells are enriched by positive selection of CD56±NK cells, for example utilizing magnetic beads conjugated to an anti-CD56 antibody (such as CD56 MicroBeads, Miltenyi Biotec, Inc., Auburn, CA). In other examples, a two-step method including negative depletion (such as T cell depletion) followed by positive selection of CD56+NK cells is used for enriching NK cells. These methods can be carried out under or adapted for Current Good Manufacturing Practice (cGMP). One of ordinary skill in the art can identify other methods that can be used to prepare an enriched population of NK cells.

Bulk NK cells or NK cell subsets isolated by additional enriching procedures, such as through the use of immuno-magnetic beads or flow sorting, may be grown in cell culture medium. In one example, the medium is Cellgro SCGM serum-free media (CellGenix, Gaithersburg, MD) containing 10% human AB serum, 50 U/mL penicillin, 50 μg/mL streptomycin, and 500 IU/mL IL-2 or in X-VIVO™ 20 media containing 10% heat inactivated human AB serum or 10% autologous serum.

The isolated NK cells can be analyzed by flow cytometry for the expression of markers such as CD56, CD16, TRAIL, FasL, NKG2D, LFA-1, perforin, and granzymes A and B. Chromium release assays can be used to assess NK cell cytotoxicity against cell targets. One of ordinary skill in the art can identify other methods to assess the isolated NK cell population (for example, purity, viability, and/or activity).

B. NK Cell Expansion

In some embodiments, enriched NK cells (typically >99% CD3 negative and >85% CD56+) are expanded in vitro. In one non-limiting example, the enriched NK cells are cultured in medium including IL-2 for up to 21 days (such as 2-5 days, 3-7 days, 6-10 days, 8-15 days, or 12-21 days). In some examples, IL-2 is included in the culture medium at about 10-2000 IU/ml (such as about 50-100 IU/ml, 100-500 IU/ml, 200-600 IU/ml, 500-1000 IU/ml, or 1000-2000 IU/ml, for example, about 10 IU/ml, 20 IU/ml, 50 IU/ml, 100 IU/ml, 200 IU/ml, 500 IU/ml, 1000 IU/ml, 1500 IU/ml, 2000 IU/ml, or more). In other examples, IL-15 is included in the culture medium at about 1-100 ng/ml (such as about 1-10 ng/ml, 5-20 ng/ml, 10-50 ng/ml, 25-75 ng/ml, or 50-100 ng/ml, for example, about 1 ng/ml, 5 ng/ml, 10 ng/ml, 20 ng/ml, 30 40 ng/ml, 50 ng/ml, 60 ng/ml, 70 ng/ml, 80 ng/ml, 90 ng/ml, or 100 ng/ml), either alone or in combination with IL-2. In additional examples, the cell culture medium may be serum-free medium (see, e.g., Moseman et al., Cytotherapy 21:S32-33, 2019). In particular examples, the enriched NK cells are also cultured in the presence of an irradiated feeder cell line (such as SMI-LCL or genetically modified K562 cells, such as K562-mb15-41BBL or K562-mbIL-21 cells).

Utilizing this technique, expansion of NK cells in the range of 200- to 1000-fold may be achieved (expanded NK cells are typically >99% CD3 negative and >90% CD56+). In some examples, the starting population of enriched NK cells is about 0.8-1.6×10 8 total NK cells, which over a 2-4 week period expand up to 1000-fold or greater in vitro. Similar numbers of NK cells have been expanded in scaled up experiments using GMP conditions. In some examples, NK cells are expanded in G-Rex® containers (Wilson Wolf, New Brighton, MN). The G-Rex®100 container supports NK expansions to doses of 2.5×10 8 NK cells/kg or higher. NK cells cultured in G-Rex®100 containers could be cultured at concentrations up to 4×10 6 NK cells/ml.

C. Modification of NK Cells

The expanded NK cells, such as those described in Section IIB are modified to reduce expression of CCR5 and optionally other genes, and may also be transduced with one or more heterologous nucleic acids to produce expression of and/or increase expression of one or more genes or transgenes of interest.

In some embodiments, a RNP including one or more sgRNAs targeting CCR5 complexed with Cas9 is introduced into the expanded NK cells. The RNP can be introduced into the NK cells by any method, including but not limited to electroporation or injection. In some examples, the RNP includes one or more sgRNAs targeting CCR5. These include, but are not limited to the nucleic acid sequences of any one of SEQ ID NOs: 1-3. In some examples, RNPs including each of SEQ ID NOs: 1-3 and Cas9 are electroporated into the expanded NK cells. In other examples, the one or more sgRNAs (such as one or more of SEQ ID NOs: 1-3) and Cas9 are introduced to the NK cells by transducing the cells with the sgRNA(s) and a nucleic acid encoding Cas9. One or ordinary skill in the art can design other sgRNAs targeting CCR5 for use with Cas9. In some examples, RNPs including one or more sgRNAs targeting one or more additional genes (e.g., CCR1, CXCR6, and or CD38) complexed with Cas9 are also introduced into the expanded NK cells, for example by electroporation. One of ordinary skill in the art can design sgRNAs targeting CCR1, CXCR6, CD38, or other genes of interest. Other methods of reducing CCR5 expression (or other genes) include use of zinc finger nucleases or antisense nucleic acid techniques, such as siRNA silencing. One of ordinary skill in the art can select appropriate techniques to reduce CCR5 gene expression (or other genes).

In some examples, the expanded NK cells are also transduced with a vector including one or more heterologous nucleic acids, such as one or more nucleic acids encoding a protein of interest (e.g., one of more of CCR7, CXCR3, CXCR4, and a CAR). In particular non-limiting examples, the vector is a lentiviral vector. Other viral vectors suitable for gene delivery to NK cells include retrovirus, adenovirus, adeno-associated virus, vaccinia virus, and fowlpox, vectors.

In particular examples, the nucleic acid(s) of interest (such as those described in Section II) is included in a lentiviral gene transfer vector. The nucleic acid(s) of interest in the transfer vector is operably linked to one or more expression control elements, such as a promoter. Exemplary promoters include constitutive promoters such as cytomegalovirus (CMV), SV40, phosphoglycerate kinase (PGK), ubiquitin C (UBC), elongation factor-1 (EFS), chicken β-action short promoter (CBH), EF-1 alpha (EF1α) promoter, or EF1α short promoter (EFS), a hybrid promoter (such as a CMV enhancer fused to chicken β-actin promoter (CAG)), or an inducible or tissue-specific promoter.

Additional expression control elements that may be included in the transfer vector include sequences that control or regulate transcription and/or translation of a nucleic acid, such as enhancers, leader sequences, transcription terminators, start and/or stop codons, internal ribosome entry sites (IRES), splicing signals, and polyadenylation signals. In examples where the vector or construct includes two (or more) heterologous nucleic acids of interest, the nucleic acids are operably linked, for example, separated by an IRES or other multicistronic element such as a P2A and/or T2A element. The vector may also contain additional elements such as packaging signals (e.g., lentivirus w packaging signal), a central polypurine tract (cPPT), a woodchuck hepatitis virus post-transcriptional regulatory element (WPRE), or a Rev Response element (RRE). In some examples, the lentivirus vector is self-inactivating.

Lentivirus vectors including one or more nucleic acids of interest can be prepared by one of ordinary skill in the art utilizing molecular biology techniques. For example, the nucleic acid of interest can be cloned into a lentivirus transfer vector. Lentivirus plasmid systems (such as 3 or 4 plasmid systems) are commercially available, for example from Clontech (Mountain View, CA), ThermoFisher Scientific (Waltham, MA), or Addgene (Cambridge, MA). In some examples, lentivirus vectors are modified to suit a particular use, such as to obtain sustained expression in NK cells.

Transduced NK cells are produced by contacting the expanded NK cells with the lentiviral particles, for example at a multiplicity of infection (MOI) of about 0.5 to 200 (such as about 0.5-5, 1-10, 5-15, 10-25, 20-50, 40-80, 60-100, 75-150, or 100-200, for example, about 0.5, 1, 2, 5, 10, 15, 20, 25, 30, 40, 50, 60, 70, 80, 90, 100, 125, 150, 175, or 200). In some examples, the NK cells are contacted with virus at an MOI of about 10-20 or an MOI of about 20. In some examples, the transduction is in the presence of one or more additional compounds, such as protamine sulfate (for example, 5-50 μg/ml, such as 5, 10, 20, 30, 40, or 50 μg/ml) or hexadimethrine bromide (e.g., Polybrene®, for example about 4-40 vg/ml, such as 4, 8, 12, 16, 20, 24, 28, 32, 36, or 40 μg/ml), or a fibronectin fragment (e.g., Retronectin®). The cells are cultured with the viral particles for about 6 hours to 5 days (for example, about 6-24 hours, 12-48 hours, 24-72 hours, 48-60 hours, or 72-96 hours), such as about 1, 2, 3, 4, or 5 days.

Following transduction of the NK cells with the lentivirus, the viral particles are removed (for example by exchanging the culture medium and optionally washing the cells). In some examples, NK cells expressing the transgene are optionally selected prior to further expansion. For example, if the transgene is expressed on the cell surface, NK cells expressing the transgene may be enriched by immuno-magnetic techniques or flow cytometry.

The modified NK cells are further expanded after modification by culturing the cells for 3-15 days or more (such as 3-10 days, 5-8 days, or 7-14 days, or more). In some examples, the NK cells are expanded in the same cell culture medium as used for the initial expansion. In some examples, the modified NK cells are also cultured in the presence of an irradiated feeder cell line (such as SMI-LCL or genetically modified K562 cells, such as K562-mb15-41BBL or K562-mbIL-21 cells). In some examples, the modified NK cells are further expanded by culturing in a medium (such as X-VIVO™ or X-VIVO™ 15 medium (Lonza, Basel, Switzerland)) including IL-2 (such as 1-1000 IU/ml, for example, 500 IU/ml IL-2). In some embodiments, one or more additional cytokines can be utilized in the expansion of the modified NK cells, including but not limited to IL-18, IL-7, IL-15, and/or IL-12. In some examples, IL-2 is included in the culture medium at about 10-2000 IU/ml (such as about 50-100 IU/ml, 100-500 IU/ml, 200-600 IU/ml, 500-1000 IU/ml, or 1000-2000 IU/ml, for example, about IU/ml, 20 IU/ml, 50 IU/ml, 100 IU/ml, 200 IU/ml, 500 IU/ml, 1000 IU/ml, 1500 IU/ml, 2000 IU/ml, or more). In other examples, IL-15 is included in the culture medium at about 1-100 ng/ml (such as about 1-10 ng/ml, 5-20 ng/ml, 10-50 ng/ml, 25-75 ng/ml, or 50-100 ng/ml, for example, about 1 ng/ml, 5 ng/ml, 10 ng/ml, 20 ng/ml, 30 40 ng/ml, 50 ng/ml, 60 ng/ml, 70 ng/ml, 80 ng/ml, 90 ng/ml, or 100 ng/ml), either alone or in combination with IL-2.

Following expansion, the modified NK cells are separated from the feeder cells (if used). The modified NK cells are washed one or more times and resuspended in an appropriate buffer or other pharmaceutically acceptable carrier, for example, for administration to a subject. In some examples, the cells are harvested and washed (for example in a buffer, such as phosphate buffered saline). The NK cells may be resuspended in a medium containing PLASMA-LYTE™ multiple electrolytes injection (Baxter Healthcare), autologous plasma, or a pharmaceutically acceptable carrier (for example, a balanced salt solution). In some examples, some or all of the modified NK cells are cryopreserved for later use.

In some examples, the modified NK cells are tested prior to administering to a subject, for example, for one or more of cell viability, tumor cell cytotoxicity, and gene expression. In additional examples, the phenotype of the modified NK cells is assessed prior to administration, such as by measuring presence and/or amount of one or more cell surface markers (such as CD56, CD16, TRAIL, FasL, NKG2D, LFA-1, perforin, or granzymes A and B), for example by flow cytometry.

### IV. Methods of Treating or Inhibiting Cancer

Disclosed herein are methods of treating a subject with cancer by administering the modified NK cells described herein to the subject. The modified NK cells described herein can be administered either to animals or to human subjects. In some embodiments, the cancer is a solid tumor. In other embodiments, the cancer is a hematological malignancy.

The modified NK cells described herein can be incorporated into pharmaceutical compositions. Such compositions typically include a population of modified NK cells and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (see, e.g., Remington: The Science and Practice of Pharmacy, The University of the Sciences in Philadelphia, Editor, Lippincott, Williams, & Wilkins, Philadelphia, PA, 21^{st} Edition, 2005). Examples of such carriers or diluents include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Actual methods for preparing administrable compositions are known or apparent to those skilled in the art and are described in more detail in such publications as Remington: The Science and Practice of Pharmacy, The University of the Sciences in Philadelphia, Editor, Lippincott, Williams, & Wilkins, Philadelphia, PA, 21^{st} Edition (2005). In one non-limiting example, the modified NK cells are suspended in PLASMA-LYTE™ multiple electrolyte solution.

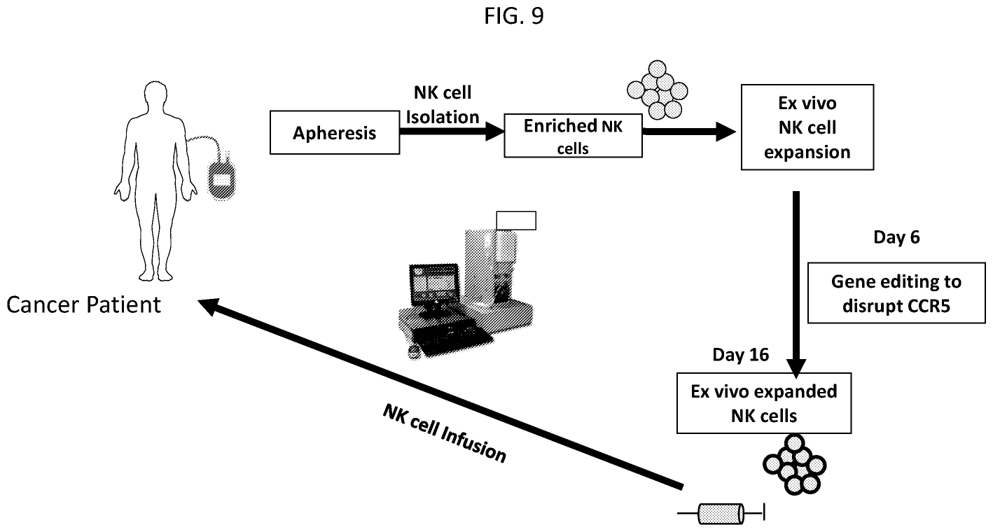

In some examples the modified NK cells are autologous to the subject being treated. In other examples, the modified NK cells are from a donor HLA-matched to the subject to be treated. An exemplary protocol for producing modified NK cells from a subject with cancer and treating the subject with the modified NK cells is shown in FIG. 9.

In some examples, the composition includes about 10⁴ to 10¹² of the modified NK cells (for example, about 10⁴-10⁷ cells, about 10⁶-10⁹ cells, or about 10⁸-10¹² cells). For example, the composition may be prepared such that about 10⁶ to 10¹⁰ modified NK cells/kg (such as about 10⁶, 10⁷, 10⁸, 10⁹, or 10¹⁰ cells/kg) are administered to a subject. The population of modified NK cells is typically administered parenterally, for example intravenously; however, injection or infusion to a tumor or close to a tumor (local administration) or administration to the peritoneal cavity can also be used. One of skill in the art can determine appropriate routes of administration.

Multiple doses of the population of modified NK cells can be administered to a subject over a period of time. For example, the population of modified NK cells can be administered daily, every other day, twice per week, weekly, every other week, every three weeks, monthly, or less frequently. A skilled clinician can select an administration schedule based on the subject, the condition being treated, the previous treatment history, and other factors.

In additional examples, the subject is also administered one or more cytokines (such as one or more of IL-2, IL-15, IL-21, and IL-12) to support survival and/or growth of the administered NK cells. The cytokine(s) are administered before, after, or substantially simultaneously with the modified NK cells. In some examples, the cytokine(s) are administered after the modified NK cells. In one specific example, the cytokine(s) is administered to the subject within about 1-8 hours (such as within about 1-4 hours, about 2-6 hours, about 4-6 hours, or about 5-8 hours) after administration of the NK cells.

In some examples, the methods include treating or inhibiting a hematological malignancy. Examples of hematological malignancies include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), T-cell large granular lymphocyte leukemia, polycythemia vera, lymphoma, diffuse large B-cell lymphoma, Hodgkin's lymphoma, non-Hodgkin's lymphoma (indolent and high grade forms), mantle cell lymphoma, follicular cell lymphoma, multiple myeloma, Waldenstrom's macroglobulinemia, heavy chain disease, myelodysplastic syndrome, hairy cell leukemia and myelodysplasia.

In other examples, the methods include treating or inhibiting a solid tumor. Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma, and retinoblastoma).

Exemplary modified NK cells that can be used to treat a subject with cancer are shown in Table 1.

TABLE 1
Exemplary modified NK cells for treating cancer

| Modified NK Cells | Target Cancer |
|---|---|
| CCR5 KO | Hematological malignancies, solid tumors |
| CCR5 KO + CXCR4 knockin | Hematological malignancies |
| CCR5 KO + CCR7 knockin | Lymphoma |
| CCR5 KO + CXCR3 knockin | Solid tumors |
| CCR5 KO + CCR1 KO | Hematological malignancies, solid tumors |
| CCR5 KO + CXCR6 KO | Hematological malignancies, solid tumors |
| CCR5KO + CD38KO + | Multiple myeloma |
| CXCR4 knockin | |

In some examples, the subject is also administered one or more additional therapies, such as chemotherapeutic agents, immunotherapeutic agents, and/or radiation therapy. One of skill in the art can select additional therapies for administration to a subject in combination with the modified NK cells described herein, for example, based on the type of cancer being treated.

In some particular examples, the subject is administered the modified NK cells and an anti-cancer monoclonal antibody. In specific, non-limiting examples, modified NK cells with reduced expression of CCR5 (and optionally also reduced expression of CD38 and/or expression of a CAR) are administered to a subject with multiple myeloma in combination with an antibody that binds to CD38 (such as daratumumab). In another particular example, modified NK cells with reduced expression of CCR5 (and optionally also increased expression of CCR7) are administered to a subject with lymphoma in combination with an antibody that binds to CD20 (such as rituximab). Additional exemplary monoclonal antibodies that can be administered to a subject in combination with the modified NK cells disclosed herein are provided in Table 2.

TABLE 2
Exemplary therapeutic monoclonal antibodies for administration
in combination with modified NK cells

| Antigen | mAb | Target Cancer |
|---|---|---|
| CD19 | GBR 401, MEDI-551 | B cell lymphoma, CLL |
| CD20 | Rituximab (RITUXAN ®), | Non-Hodgkin's lymphoma |
| | ofatumumab (ARZERRA ®), | |
| | veltuzumab | |
| | Ibritumomab tiuxetan | Lymphoma |
| | (ZEVALIN ®), obinutuzumab, | |
| | ublituximab, tositumomab | |
| | (BEXXAR ®), ocaratuzumab | |
| CD22 | Narnatumab, inotuzumab | Cancer |
| | ozogamicin | |
| CD30 | Brentuximab vedotin | Hodgkin's lymphoma |
| | (ADCETRIS ®), iratumumab | |
| CD33 | Gemtuzumab ozogamicin | Acute myelogenous leukemia |
| | (MYLOTARG ®), lintuzumab, | |
| CD37 | Otlertuzumab | Cancer cells |
| CD38 | Daratumumab | Multiple myeloma |
| Antigen | mAb | Target Cancer |
| CD40 | Lucatumumab, dacetuzumab | Multiple myeloma, non- |
| | | Hodgkin's or Hodgkin's |
| | | lymphoma |
| CD52 | Alemtuzumab (CAMPATH ®, | Chronic lymphocytic leukemia |
| | MABCAMPATH ®, | |
| | CAMPATH-1H ®) | |
| CD56 | Lorvotuzumab mertansine | Small-cell lung cancer, ovarian |
| | | cancer |
| CD70 | Vorsetuzumab mafodotin | Renal cell carcinoma |
| CD74 | Milatuzumab | Multiple myeloma |
| CD140 | Tovetumab | cancer |
| EpCAM | IGN101, oportuzumab | Epithelial tumors (breast, colon |
| | monatox, tucotuzumab | and lung) |
| | celmoleukin, adecatumumab | |
| CEA | Labetuzumab (CEA-CIDE ®) | Breast, colon and lung tumors |
| gpA33 | huA33 | Colorectal carcinoma |
| mesothelin | Amatuximab | Cancer cells |
| α-fetoprotein | ⁹⁰Y-tacatuzumab tetraxetan | Tumor cells |
| IL-6 | Siltuximab | Metastatic renal cell cancer, |
| | | prostate cancer, and Castleman's |
| | | disease |
| Mucins | Pemtumomab | Breast, colon, lung and ovarian |
| | (THERAGYN ®), | tumors |
| | cantuzumab mertansine, ⁹⁰Y | |
| | clivatuzumab tetraxetanand, | |
| | oregovomab (OVAREX ®) | |
| PDGFR- | Olaratumab | Solid tumors |
| alpha | | |
| TAG-72 | CC49 (minretumomab) | Breast, colon and lung tumors |
| CAIX | Girentuximab, cG250 | Renal cell carcinoma |
| PSMA | J591 | Prostate carcinoma |
| Folate- | MOv18 and MORAb-003 | Ovarian tumors |
| binding | (farletuzumab) | |
| protein | | |
| Scatter factor | Onartuzumab | Cancer cells |
| receptor | | |
| kinase | | |
| Gangliosides | 3F8, ch14.18, KW-2871 | Neuroectodermal tumors and |
| (e.g., GD2, | | some epithelial tumors |
| GD3 and | | |
| GM2) | | |
| Cytokeratin | ^{99m}Tc- Votumumab | Colorectal tumors |
| | (HUMASPECT ®) | |
| Frizzled | Vantictumab | Cancer |
| receptor | | |
| Le^{y} | hu3S193, IgN311 | Breast, colon, lung and prostate |
| | | tumors |
| VEGF | Bevacizumab (AVASTIN ®) | Tumor vasculature |
| VEGFR | IM-2C6, CDP791 | Epithelium-derived solid tumors |
| Integrin | Etaracizumab (ABEGRIN ®), | Tumor vasculature |
| αVβ3 | intetumumab | |
| Integrin α5β1 | Volociximab | Tumor vasculature |
| EGFR | Cetuximab (ERBITUX ®), | Glioma, lung, breast, colon, and |
| | panitumumab (VECTIBIX ®), | head and neck tumors |
| | nimotuzumab, necitumumab, | |
| | zalutumumab, imgatuzumab, | |
| | matuzumab, 806 | |
| EGFL7 | Parsatuzumab | Cancer cells |
| ERBB2 | Trastuzumab (HERCLON ®; | Breast, colon, lung, ovarian and |
| | HERCEPTIN ®), pertuzumab | prostate tumors |
| | (PERJETA ®; | |
| | OMNITARG ®) | |
| ERBB3 | Duligotumab, MM-121 | Breast, colon, lung, ovarian and |
| | | prostate, tumors |
| Fibronectin | Radretumab | antineoplastic |
| HGF | Rilotumumab, ficlatuzumab | Solid tumors |
| HER3 | Patritumab | Cancer |
| MET | AMG 102, METMAB, SCH | Breast, ovary and lung tumors |
| | 900105 | |
| IGF1R | Cixutumumab, dalotuzumab, | Glioma, lung, breast, head and |
| | figitumumab, ganitumab, | neck, prostate and thyroid cancer |
| | robatumumab, teprotumumab, | |
| | AVE1642, IMC-A12, MK- | |
| | 0646, R1507, and CP 751871 | |
| IGLF2 | Dusigitumab | |
| EPHA3 | KB004, IIIA4 | Lung, kidney and colon tumors, |
| | | melanoma, glioma and |
| | | hematological malignancies |
| FR-alpha | Farletuzumab | Ovarian cancer |
| phosphatidylserine | Bavituximab | Cancer cells |
| Syndecan 1 | Indatuximab ravtansine | |
| SLAMF7 | Elotuzumab | Multiple myeloma |
| (CD319) | | |
| TRAILR1 | Mapatumumab (HGS-ETR1) | Colon, lung and pancreas tumors |
| | | and hematological malignancies |
| TRAILR2 | Conatumumab, lexatumumab, | Cancer |
| | mapatumumab, tigatuzumab, | |
| | HGS-ETR2, CS-1008 | |
| RANKL | Denosumab (XGEVA ®) | Prostate cancer and bone |
| | | metastases |
| FAP | Sibrotuzumab, and F19 | Colon, breast, lung, pancreas, and |
| | | head and neck tumors |
| vimentin | Pritumumab | Brain cancer |
| Tenascin | 81C6 | Glioma, breast and prostate |
| | | tumors |

### EXAMPLES

The following examples are provided to illustrate certain particular features and/or embodiments. These examples should not be construed to limit the disclosure to the particular features or embodiments described.

### Example 1

### Materials and Methods

Primary cells and cell lines: Peripheral blood mononuclear cells (PBMCs) from healthy donors were isolated using Lymphocyte Separation Medium (MP Biomedicals). NK cells were isolated by magnetic bead separation, CD3-depleted and CD56-selected (Miltenyi), and expanded in vitro for 14-16 days in G-Rex (Wilson Wolf Manufacturing) flasks with irradiated human SMI-EBV-LCL feeder cells at a ratio of 1:10 or with GE-K562 cells containing membrane bound IL-21 and 41BBL (K562. mblL-21.41BBL) at a ratio of 1:2. SMI-EBV-LCLs were established in the NHLBI/NIH by Dr. Richard Childs' lab, and K562.mbIL-21.41BBL cell line was generously provided by Dr. Katy Rezvani from MD Anderson Cancer Center. Cell lines were propagated in RPMI 1640 supplemented with 10% heat-inactivated FBS (Sigma-Aldrich). NK cells were cultured in X-vivo 20 media (Lonza) containing 10% human AB serum (Sigma), 1% Glutamax (Gibco), and 500 U/mL IL-2. For NK cell expansion, fresh media was supplied to cells starting on day 5 and then every 2-3 days until the cells were harvested for use in experiments. During NK cell expansion, cells were maintained to have a consistent cell concentration of 0.8-1.5×10⁶/mL between all samples.

Flow cytometry: Directly following NK cell isolation from healthy donor PBMCs (n=6), population purity was defined by flow cytometry using the following panel: CD56-PE Cy7 (BD biosciences, clone NCAM16), CD3-FITC (BD Biosciences, clone SK7), CD19-PE (BD Biosciences, clone SJ25C1), and CD14-PB (Biolegend, clone M5E2). Freshly isolated NK cells from each donor that were rested overnight in NK media without IL-2 and paired samples that were expanded for 16 days ex vivo were stained using the following antibody reagents: CXCR6 (BD Biosciences, clone 13B 1E5), CCR1 (Biolegend, clone 5F10B29), CCR5 (Biolegend, clone J418F1), CD151 (BD Biosciences, clone 14A2.H1), CD11A (BD Biosciences, clone HI111), CD49D (Biolegend 9F10), CD2 (BD Biosciences, clone S5.2), DNAM1 (Biolegend, clone 10E5), CD18 (Biolegend, clone CBR LFA 1/2), CD9 (BD Biosciences, clone M-L13), CD29 (Biolegend, clone TS2/16), CCR6 (BD Biosciences, clone 11A9), CXCR3 (Biolegend, clone G025H7), CD44 (Biolegend, clone IM7), PSGL1 (Biolegend, clone KPL-1), CXCR4 (Biolegend, clone 12G5), CCR7 (Biolegend, clone 4B12), CXCR1 (Biolegend, clone 8F1), CX3CR1 (Biolegend, clone 2A0-1). Following 30 minutes of antibody staining at 4° C., cells were washed with PBS containing 10% FBS and 2 mM EDTA and fixed with PBS containing 1% paraformaldehyde. Flow cytometry was conducted using an LSRFortessa cytometer (BD biosciences) and data was analyzed using FlowJo (BD biosciences, v.9).

RNA-sequencing and analysis: RNA was isolated from 1×10⁷ fresh NK cells and 1×10⁷ ex vivo expanded NK cells after 16 days of culture (RNAeasy® Minikit, Qiagen). RNA isolates were submitted to Novagene (Sacramento, CA) for sequencing and analysis. Sequencing libraries were prepared, and quality of the libraries was assessed by Qubit (Thermo Fischer Scientific, Wilmington, DE), 2100 Bioanalyzer (Aligent, Santa Clara, CA), and qPCR. Qualifying samples were sequenced by HiSeq (Illumina, San Diego CA), and 150 bp paired-end reads were generated at a depth of 20×. Sequencing-generated fastq files were checked for read quality and mapping rate with FastQC (0.11.8), clean reads were aligned with STAR (v2.5) to hg38 reference genome. Gene expression level was determined using HTSeq (v0.6.1) and DESeq2 R package (v2_1.6.3). Data is reported as Fragments Per Kilobase of exon model per Million mapped reads (FPKM).

Construction of lymphocyte trafficking dataset: A large dataset of genes that are relevant to in vivo cellular trafficking (Table 3) was constructed based on compiling genes lists from several gene ontology (GO) categories, using the QuickGO tool (EMBL-EBI). The custom table was built using the GO categories listed in Table 4. The list was filtered to only include Homo sapiens genes that had the qualifiers ‘part of,’ ‘involved in,’ or ‘enables’ for the aspects ‘molecular function,’ ‘cellular component,’ and ‘biological process.’ The list was then merged with data output from DESeq2, to create a dataset to include FPKM values, fold change in expression due to NK cell expansion, and adjusted significance for each gene. Genes that had an average FPKM of <1 across all samples were eliminated from analysis. Table 4 serves as metadata for Table 3.

Identification of soluble factors in ex vivo NK cell expansions: Supernatants were collected from NK expansion cultures as described above on days 2, 3, 4, and 5 and stored at −20° C. Concentrations of analytes were measured using a Mesoscale Discovery multiplex panel (Mesoscale Diagnostics). Supernatants were thawed and assessed for the presence of IFN-γ, TNF-α, CCL3, IL-10, VEGF, granulocyte/macrophage-CSF (GM-CSF), SDF-1α, with IL-2 used as a positive control.

Cytokine exposure assay: NK cells were freshly isolated from healthy donor PBMCs using Rosette Sep (Stem cell Technologies). Fresh NK cells were resuspended in NK media without IL-2 at a concentration of 5×10⁵ cells/mL in a 24-well cell culture plate (Corning). Next, 20 ng/mL rh (recombinant human) G-CSF (Neupogen), 200 U/mL rhIL-2 (Tecin), 50 ng/mL rhTNF-α (R&D Systems), 200 ng/mL rhIFN-γ (R&D Systems), 20 ng/mL rhIL-4 (R&D Systems), 20 ng/mL rhIL-Systems), 20 ng/mL rhGM-CSF (R&D Systems), 20 ng/mL rhIL-10 (R&D Systems), 5 ng/mL rhTGF-β (R&D Systems), 50 ng/mL rhCCL5 (R&D Systems), 50 ng/mL rhCCL3 (R&D Systems), 50 ng/mL rhVEGF165 (R&D Systems), or 200 ng/mL rhSDF-1α (R&D Systems) were individually added to each well. NK cells in media with no cytokine were used as the control. All samples were incubated for 24 hours in 37° C. 6.5% CO₂. Then, 1×10⁵ cells from each sample were collected and stained for flow cytometry-based identification of CCR1, CCR5, CXCR4, and CXCR6 surface expression.

In vivo trafficking of CCR5 knockout (KO) NK cells: Gene KO Kit v2 (Synthego) was used to specifically target CCR5 in primary NK cells. Primary NK cells were expanded ex vivo using irradiated SMI-EBV-LCL feeder cells as described above. NK cells were collected on day 6 and were electroporated (Lonza 4D) with CIRSPR/Cas9 ribonucleoprotein (RNP) complexes. Three synthetic guide RNAs were used in a single electroporation: UUUUGCAGUUUAUCAGGAUG (SEQ ID NO: 1), AAAACAGGUCAGAGAUGGCC (SEQ ID NO: 2), and UGUAUUUCCAAAGUCCCACU (SEQ ID NO: 3), with each sequence preceding a Synthego modified EZ scaffold. Following CRISPR/Cas9 KO of CCR5, NK cells were maintained in expansion culture. On day 14, CCR5 KO NK cells and non-modified NK cells were collected, rinsed 2× with PBS and suspended in PBS for IV infusion into 8-10-week old NSG mice (Jackson Laboratories). Mice received 1×10⁷ CCR5 KO NK cells (n=4) or non-modified NK cells (n=3) and then were harvested 24 hours following injection; blood, bone marrow, liver, and spleens were collected. Cells were manually dissociated and evaluated for CD56, HLA class 1, and CCR5 surface expression by flow cytometry; 100 μl of each sample was acquired with an LSR Fortessa cytometer and the data was analyzed with Flowjo. Live/dead-CD56+ HLA class 1+ events were acquired from each mouse. The percentage of events within each organ was determined as a fraction of all of the events acquired per mouse. This experiment was approved and conducted in accordance with the NIH animal study advisory committee guidelines.

Analysis of statistics: Statistical analysis of RNA-seq data was output by DESeq2 R package (2_1.6.3). Benjamini and Hochberg's false discovery rate approach was utilized to calculate the adjusted p values for each event. Genes with an adjusted p value of <0.05 by were determined to be differentially expressed. All other data were analyzed with Prism 7.0b software (GraphPad Software Inc.), using the two-tailed paired T-test, Student's T-test, the Welch's-T-test to report significant differences between groups. The appropriate test was chosen based on data distribution, variances, and experimental set up.

### Example 2

### Transcriptional Landscape of NK Cells Expanded Ex Vivo with Feeder Cells Compared to Freshly Isolated NK Cells

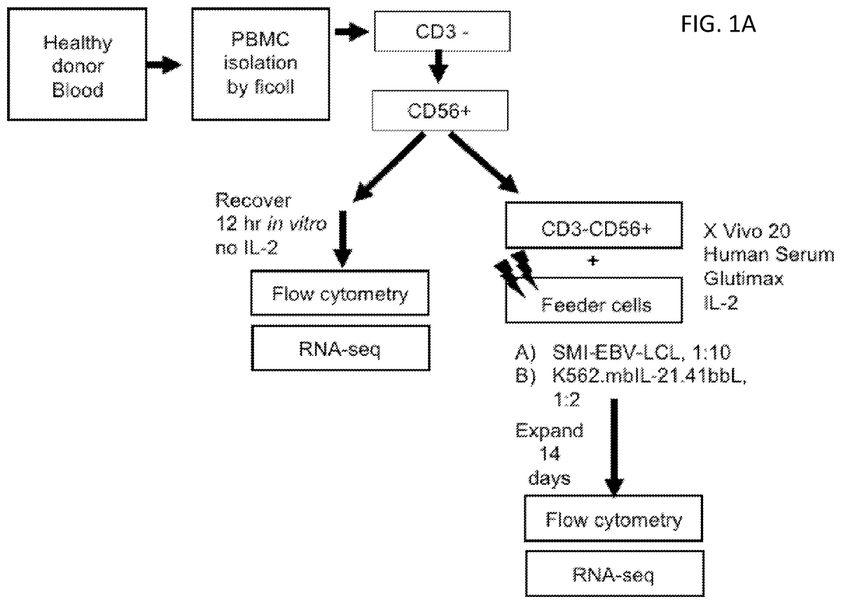

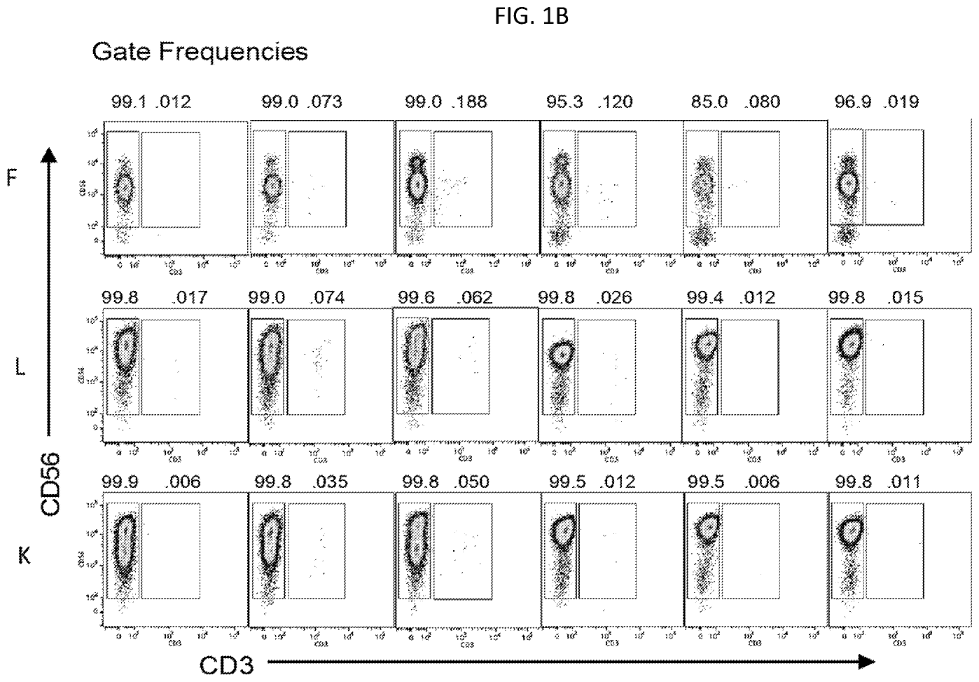

Ex vivo activation and expansion of primary NK cells using irradiated feeder cells is commonly utilized to produce large numbers of highly cytotoxic NK cells for clinical use. Therefore, a detailed analysis using flow cytometry and RNA-seq was performed to characterize phenotypic and gene transcription changes associated with ex vivo expansion of NK cells using two feeder lines that are currently or have previously been used in clinical trials. These feeder lines are K562.mbIL-21.41BBL (e.g., NCT01619761, NCT01729091, NCT01787474, NCT01904136, NCT02271711, NCT02573896, NCT02727803, NCT02809092, NCT03019640, NCT03348033, NCT03420963, NCT03579927), and EBV-LCLs (e.g., NCT00720785) (FIG. 1A). The majority of freshly isolated NK cells and all of the ex vivo expanded NK cell samples utilized in this study were confirmed to be a highly viable and purified CD56+NK cell population devoid of T-cells (less than 0.2% T cell contamination; n=6 HDs) (FIG. 1B).

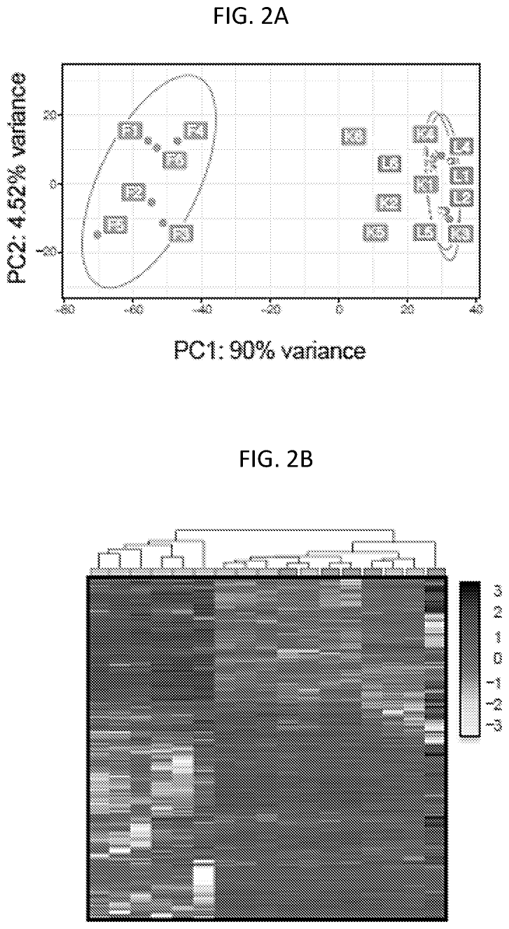

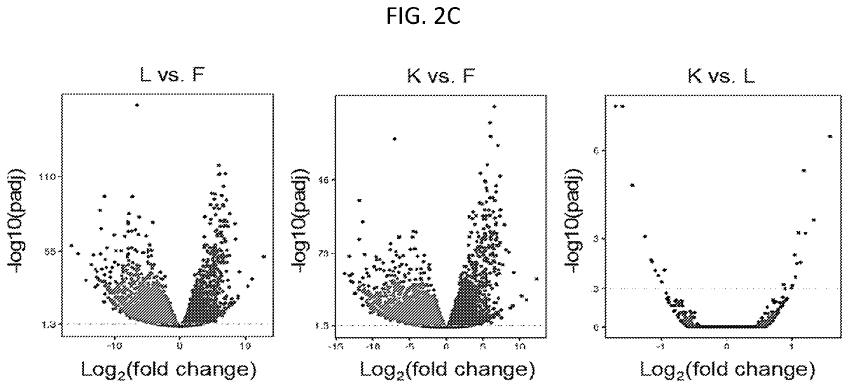

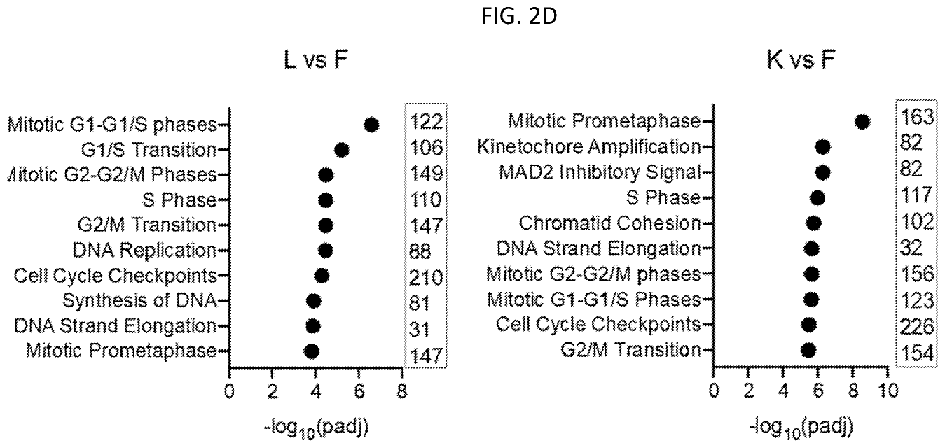

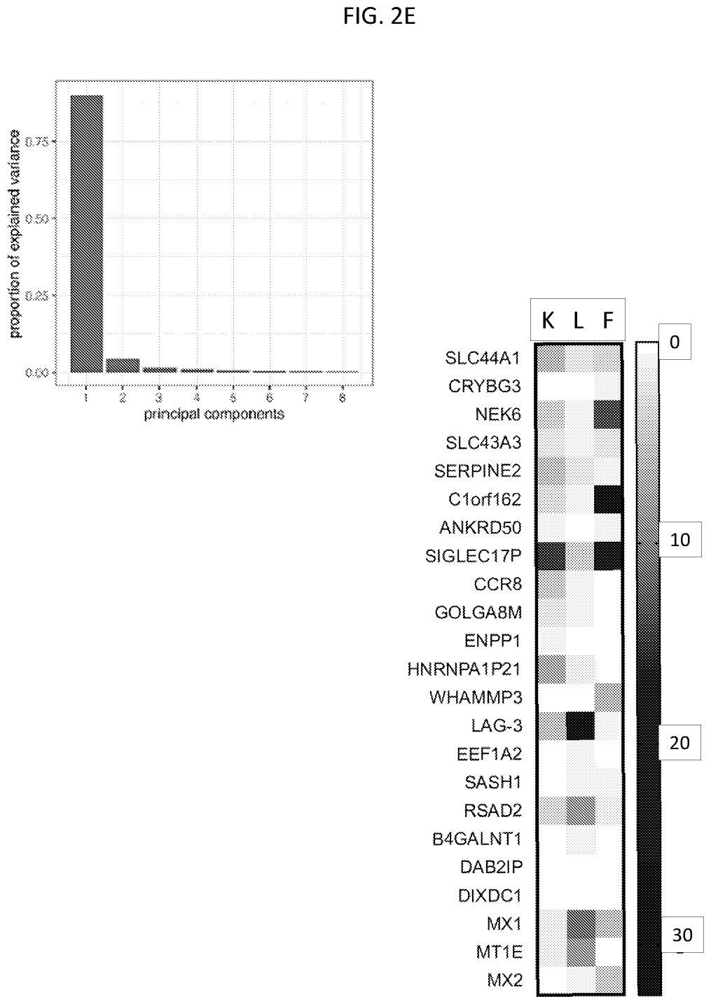

As determined by FastQC quality check analysis of the sequenced samples, >94% of samples achieved a quality score of >30 and all samples achieved a mapping rate of >95%. Principal component analysis (PCA) and an unsupervised heat map clustering analysis of ex vivo expanded and fresh NK cell samples exhibited distinct gene expression properties of these cell populations (FIGS. 2A and 2B). PC1 accounted for 90% of the genetic differences between fresh and expanded NK cells, and represented gene sets that are involved with cell cycle and proliferation. PC2 accounted for approximately 5% of differences that were exhibited amongst the fresh NK cell samples and is likely due to don0r-specific genetic variability; all other components accounted for less than 1% of variance. The outlying donor in the fresh NK cell cohort was defined to have only 85% pure CD56+NK cells, as opposed to other samples that were >95% pure CD56+(FIG. 1B). However, after ex vivo expansion, NK cell purity became >99% CD56+ and PC3 differences were diminished.

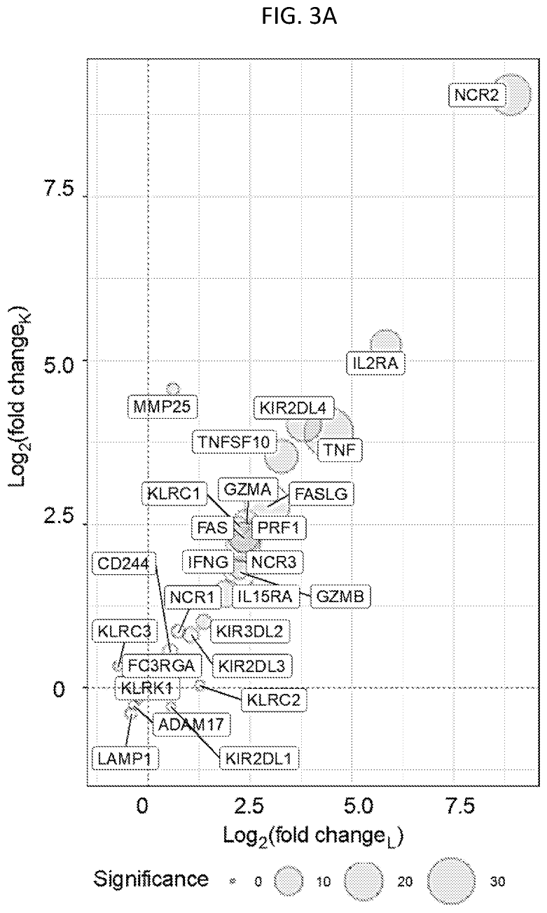

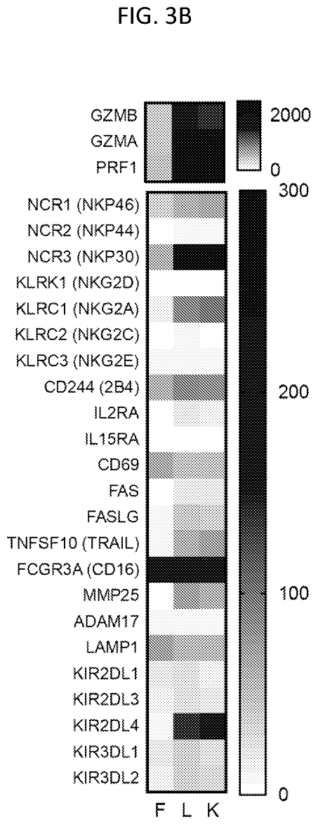

Compared to fresh NK cells, ex vivo expansion of NK cells with SMI-EBV-LCL cells led to an upregulation of 4257 genes and a downregulation of 7661 genes, using an adjusted p value threshold of <0.05 assigned by DESeq2 algorithm. In a similar fashion, ex vivo expansion of NK cells using GE-K562 led to an upregulation of 4981 genes and a downregulation of 8151 genes (FIG. 2C). Populations of NK cells expanded with either feeder cell line were transcriptionally similar; significant differential gene expression that was observed included the upregulation of 13 genes and the downregulation of 10 genes, with each of these genes having low transcript abundance (FPKM <30) (FIGS. 2D and 2E). Gene transcription was significantly higher in ex vivo expanded NK cells for GZMB, GZMA, PRF1, FAS, FASL, TNFSF10, 2B4, NCR1, NCR3 (FIGS. 3A and 3B).

### Example 3

### Transcriptional Shifts of Genes Related to Cell Trafficking in Ex Vivo Expanded NK Cells

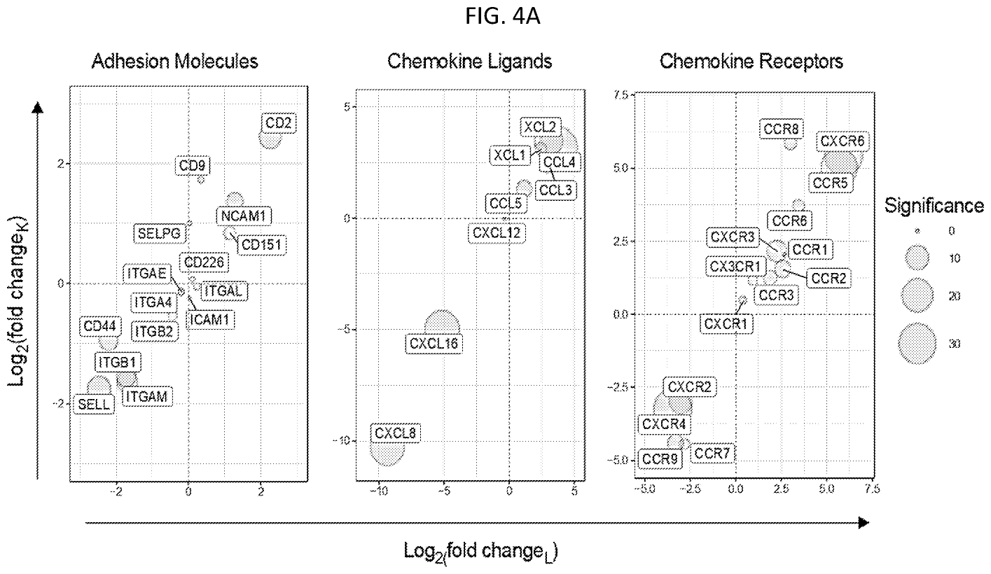

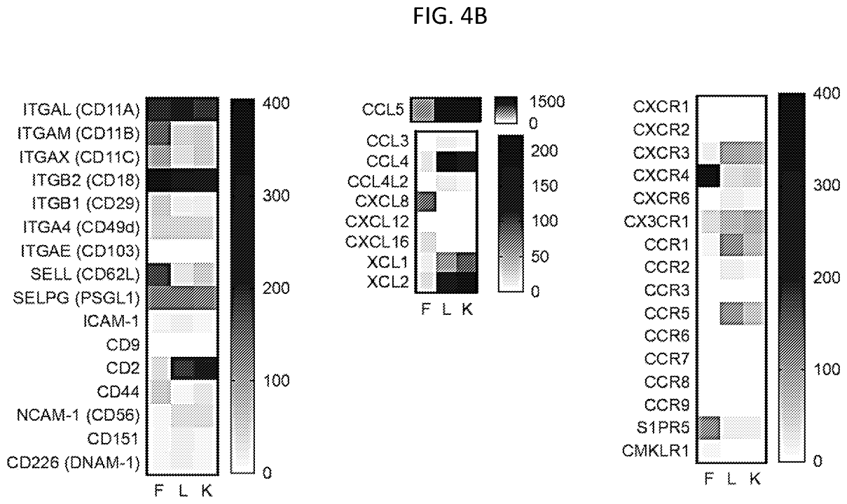

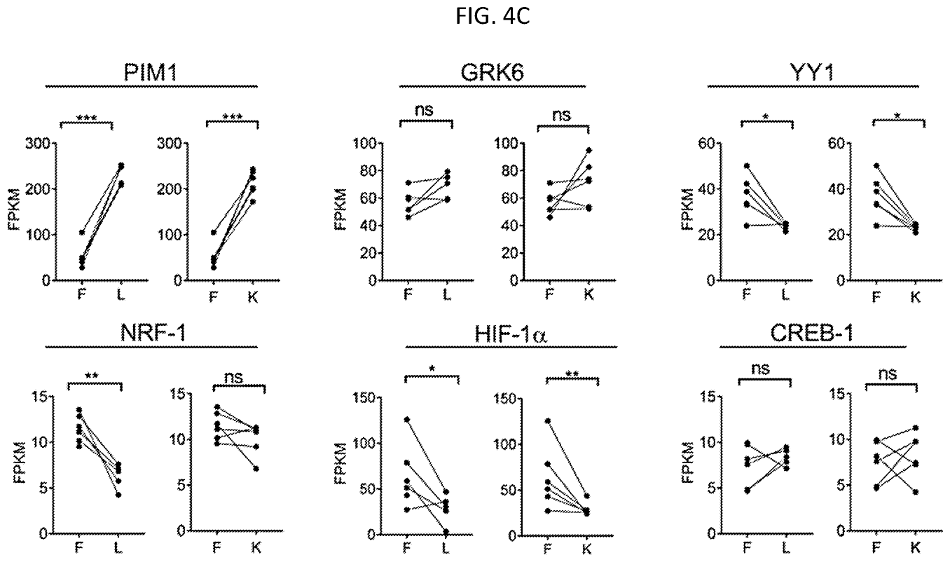

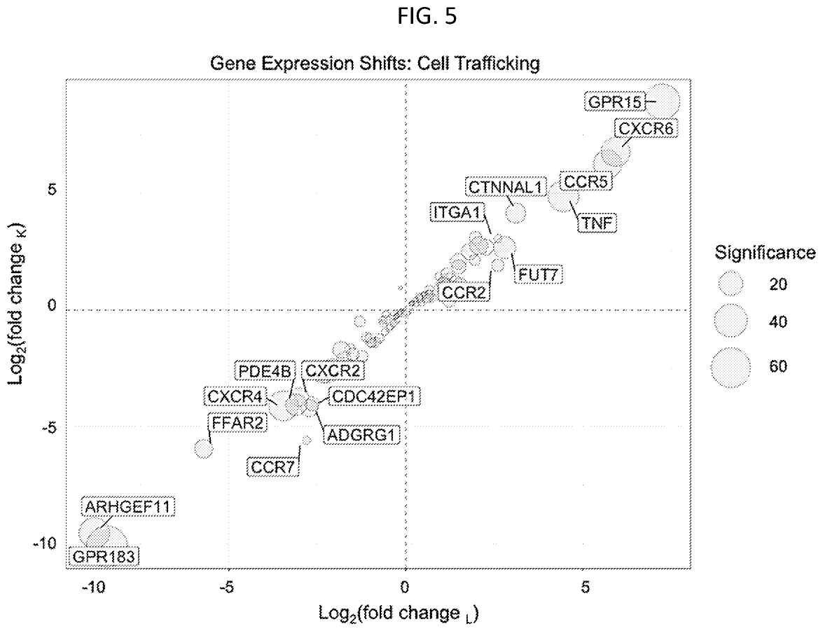

Given the homing of adoptively transferred NK cells to sites of malignancy is likely an important determinant of therapeutic efficacy, efforts were focused to detail differential gene expression of chemotactic and adhesion molecules known to regulate cell trafficking. Among the largest and most statistically significant differences observed in the dataset were the downregulation of CXCR4 and the upregulation of CXCR3, CXCR6, CCR5 and CCR1 in expanded NK cells compared to fresh NK cells (FIGS. 4A and 4B). Specifically, CXCR4 was downregulated >10-fold (adjusted p=1.14E-33) and CXCR6 was upregulated approximately 60-fold (adjusted p=3.00E-31) on SMI-EBV-LCL-expanded NK cells. Transcription of CCR5 and CCR1 were also substantially upregulated in expanded NK cells compared to fresh NK cells (>50-fold, adjusted p=8.76E-30 and >4-fold, adjusted p=9.3E-5, respectively). Remarkably, a substantial increase in transcription of chemokine ligands XCL1, XCL2, CCL3, CCL4, and CCL5, and a significant loss in transcription of CXCL8, CXCL16, and SIPR5 were also observed (FIGS. 4A and 4B).

Among adhesion molecules, the most notable changes in transcription occurring with ex vivo NK cell expansion were a decrease in CD62L (approximately 5-fold, adjusted p=1.82E-11) and a strong increase in CD2 (approximately 4-fold, p=4.0E-11) (FIGS. 4A and 4B). Genes that are responsible for the downstream signaling of chemokine receptors including genes involved in MAP, RHO, and PI3 kinase signal cascade remained unchanged (FIG. 2, Table 3). Changes in gene expression of CXCR4 transcriptional regulators and post-translational regulators were also assessed. Of these genes, PIM-1 and GRK6 were increased in expanded NK cells, NRF-1 and YY-1 gene transcription was reduced in expanded NK cells, and CREB-1 expression was unchanged in expanded NK cells compared to fresh NK cells (FIG. 4C).

TABLE 4
GO categories used to build Table 3.

| GO TERM | GO NAME |
|---|---|
| GO:0002029 | desensitization of G protein-coupled receptor signaling pathway |
| GO:0002032 | desensitization of G protein-coupled receptor signaling pathway by arrestin |
| GO:0002232 | leukocyte chemotaxis involved in inflammatory response |
| GO:0002407 | dendritic cell chemotaxis |
| GO:0002408 | myeloid dendritic cell chemotaxis |
| GO:0002522 | leukocyte migration involved in immune response |
| GO:0002548 | monocyte chemotaxis |
| GO:0002551 | mast cell chemotaxis |
| GO:0004687 | myosin light chain kinase activity |
| GO:0004703 | G protein-coupled receptor kinase activity |
| GO:0004705 | JUN kinase activity |
| GO:0004707 | MAP kinase activity |
| GO:0004950 | chemokine receptor activity |
| GO:0007266 | Rho protein signal transduction |
| GO:0008417 | fucosyltransferase activity |
| GO:0010818 | T cell chemotaxis |
| GO:0016262 | protein N-acetylglucosaminyltransferase activity |
| GO:0016493 | C-C chemokine receptor activity |
| GO:0016494 | C-X-C chemokine receptor activity |
| GO:0016495 | C-X3-C chemokine receptor activity |
| GO:0017083 | 4-galactosyl-N-acetylglucosaminide 3-alpha-L-fucosyltransferase activity |
| GO:0030593 | neutrophil chemotaxis |
| GO:0030595 | leukocyte chemotaxis |
| GO:0032488 | Cdc42 protein signal transduction |
| GO:0033622 | integrin activation |
| GO:0035685 | helper T cell diapedesis |
| GO:0035696 | monocyte extravasation |
| GO:0035705 | T-helper 17 cell chemotaxis |
| GO:0035754 | B cell chemotaxis |
| GO:0036336 | dendritic cell migration |
| GO:0038036 | sphingosine-1-phosphate receptor activity |
| GO:0038116 | chemokine (C-C motif) ligand 21 signaling pathway |
| GO:0038163 | thrombopoietin-mediated signaling pathway |
| GO:0045123 | cellular extravasation |
| GO:0046920 | alpha-(1->3)-fucosyltransferase activity |
| GO:0046921 | alpha-(1->6)-fucosyltransferase activity |
| GO:0048245 | eosinophil chemotaxis |
| GO:0048246 | macrophage chemotaxis |
| GO:0048247 | lymphocyte chemotaxis |
| GO:0050900 | leukocyte migration |
| GO:0050901 | leukocyte tethering or rolling |
| GO:0050904 | diapedesis |
| GO:0070098 | chemokine-mediated signaling pathway |
| GO:0071621 | granulocyte chemotaxis |
| GO:0071674 | mononuclear cell migration |
| GO:0072676 | lymphocyte migration |
| GO:0072677 | eosinophil migration |
| GO:0072678 | T cell migration |
| GO:0072679 | thymocyte migration |
| GO:0072683 | T cell extravasation |
| GO:0097021 | lymphocyte migration into lymphoid organs |
| GO:0097530 | granulocyte migration |
| GO:0097531 | mast cell migration |
| GO:0120117 | T cell meandering migration |
| GO:1905517 | macrophage migration |
| GO:1990266 | neutrophil migration |

TABLE 3
Genes identified relevant to in vivo cellular trafficking

| | | | | | L vs F | K vs F | L vs | K vs | F | | L | | K | |
|---|---|---|---|---|---|---|---|---|---|---|---|---|---|---|
| Gene | | Gene | | | log2Fold | log2Fold- | F | F | FPKM | F | FPKM | L | FPKM | K |
| Name | Gene ID | Description | QUALIFIER | GO NAME | Change | Change | padj | padj | AVG | STDEV | AVG | STDEV | AVG | STDEV |
| ADAM8 | ENSG00000151651 | ADAM_metallopeptidase_domain_8 | involved_in | leukocyte | −0.588 | −0.739 | 0.099 | 0.000 | 110.481 | 7.506 | 72.909 | 34.596 | 67.321 | 8.478 |
| | | | | migration | | | | | | | | | | |
| | | | | involved in | | | | | | | | | | |
| | | | | inflammatory | | | | | | | | | | |
| | | | | response | | | | | | | | | | |
| ADGRG1 | ENSG00000205336 | adhesion_G_protein-coupled_{—} | involved_in | Rho protein | −2.727 | −3.354 | 0.000 | 0.000 | 77.856 | 34.383 | 10.981 | 6.518 | 7.251 | 3.549 |
| | | receptor_G1 | | signal | | | | | | | | | | |
| | | | | transduction | | | | | | | | | | |
| ADRBK1 | ENSG00000173020 | adrenergic_beta_receptor_kinase_1 | involved_in | desensitization | 0.061 | −0.106 | 0.851 | 0.598 | 124.195 | 17.471 | 130.217 | 26.894 | 116.929 | 7.797 |
| | | | | of G protein- | | | | | | | | | | |
| | | | | coupled receptor | | | | | | | | | | |
| | | | | signaling | | | | | | | | | | |
| | | | | pathway | | | | | | | | | | |
| ADRBK2 | ENSG00000100077 | adrenergic_beta_receptor_kinase_2 | enables | G protein- | −0.817 | −1.068 | 0.128 | 0.024 | 5.923 | 5.112 | 3.545 | 0.983 | 2.947 | 0.796 |
| | | | | coupled receptor | | | | | | | | | | |
| | | | | kinase activity | | | | | | | | | | |
| ANO6 | ENSG00000177119 | anoctamin_6 | involved_in | dendritic cell | −0.652 | −0.354 | 0.065 | 0.241 | 15.160 | 3.596 | 10.025 | 3.500 | 12.035 | 3.055 |
| | | | | chemotaxis | | | | | | | | | | |
| ARHGAP1 | ENSG00000175220 | Rho_GTPase_activating_protein_1 | involved_in | Rho protein | 0.510 | 0.503 | 0.116 | 0.019 | 16.669 | 3.933 | 23.636 | 7.344 | 23.762 | 3.532 |
| | | | | signal | | | | | | | | | | |
| | | | | transduction | | | | | | | | | | |
| ARHGAP4 | ENSG00000089820 | Rho_GTPase_activating_protein_4 | involved_in | Rho protein | −0.464 | −0.620 | 0.109 | 0.000 | 62.799 | 7.865 | 45.380 | 12.312 | 41.229 | 2.089 |
| | | | | signal | | | | | | | | | | |
| | | | | transduction | | | | | | | | | | |
| ARHGAP5 | ENSG00000100852 | Rho_GTPase_activating_protein_5 | involved_in | Rho protein | −0.175 | −0.075 | 0.714 | 0.768 | 2.212 | 0.563 | 1.998 | 0.912 | 2.096 | 0.289 |
| | | | | signal | | | | | | | | | | |
| | | | | transduction | | | | | | | | | | |
| ARHGDIA | ENSG00000141522 | Rho_GDP_dissociation_inhibitor_{—} | involved_in | Rho protein | 0.956 | 0.779 | 0.001 | 0.000 | 93.690 | 16.746 | 182.717 | 45.360 | 162.843 | 13.392 |
| | | (GDI)_alpha | | signal | | | | | | | | | | |
| | | | | transduction | | | | | | | | | | |
| ARHGDIB | ENSG00000111348 | Rho_GDP_dissociation_inhibitor_{—} | involved_in | Rho protein | 0.428 | 0.361 | 0.055 | 0.070 | 407.107 | 43.845 | 555.537 | 92.978 | 525.799 | 84.039 |
| | | (GDI)_beta | | signal | | | | | | | | | | |
| | | | | transduction | | | | | | | | | | |
| ARHGEF1 | ENSG00000076928 | Rho_guanine_nucleotide_exchange_{—} | involved_in | Rho protein | −0.978 | −1.104 | 0.001 | 0.000 | 118.292 | 23.438 | 58.864 | 21.899 | 55.259 | 12.313 |
| | | factor_1 | | signal | | | | | | | | | | |
| | | | | transduction | | | | | | | | | | |
| ARHGEF11 | ENSG00000132694 | Rho_guanine_nucleotide_exchange_{—} | involved_in | Rho protein | −8.784 | −7.627 | 0.000 | 0.000 | 4.699 | 4.649 | 0.009 | 0.009 | 0.022 | 0.016 |
| | | factor_11 | | signal | | | | | | | | | | |
| | | | | transduction | | | | | | | | | | |
| ARHGEF12 | ENSG00000196914 | Rho_guanine_nucleotide_exchange_{—} | involved_in | Rho protein | 0.209 | 0.238 | 0.528 | 0.408 | 4.539 | 1.142 | 5.317 | 1.717 | 5.315 | 1.579 |
| | | factor_12 | | signal | | | | | | | | | | |
| | | | | transduction | | | | | | | | | | |
| ARHGEF3 | ENSG00000163947 | Rho_guanine_nucleotide_exchange_{—} | involved_in | Rho protein | 1.094 | 1.056 | 0.003 | 0.002 | 20.052 | 11.550 | 42.466 | 9.255 | 41.348 | 12.707 |
| | | factor_3 | | signal | | | | | | | | | | |
| | | | | transduction | | | | | | | | | | |
| ARRB2 | ENSG00000141480 | arrestin_beta_2 | involved_in | desensitization | −0.254 | −0.354 | 0.486 | 0.230 | 90.385 | 29.431 | 77.616 | 11.963 | 72.681 | 10.073 |
| | | | | of G protein- | | | | | | | | | | |
| | | | | coupled receptor | | | | | | | | | | |
| | | | | signaling | | | | | | | | | | |
| | | | | pathway by | | | | | | | | | | |
| | | | | arrestin | | | | | | | | | | |
| B4GALT1 | ENSG00000086062 | UDP-Gal:betaGlcNAc_beta_14-_{—} | involved_in | leukocyte | −2.232 | 2.121 | 0.000 | 0.000 | 88.157 | 23.634 | 19.077 | 2.065 | 20.515 | 1.382 |
| | | galactosyltansferase_polypeptide_1 | | migration | | | | | | | | | | |
| C15orf62 | ENSG00000188277 | chromosome_15_open_reading_frame_62 | involved_in | Rho protein | −1.844 | −2.083 | 0.000 | 0.000 | 3.907 | 1.200 | 1.028 | 0.807 | 0.914 | 0.149 |
| | | | | signal | | | | | | | | | | |
| | | | | transduction | | | | | | | | | | |
| CCR2 | ENSG00000121807 | chemokine_(C-C_motif)_receptor_2 | enables | C-C chemokine | 2.592 | 1.528 | 0.000 | 0.039 | 2.859 | 2.656 | 19.549 | 13.505 | 8.927 | 6.083 |
| | | | | receptor activity | | | | | | | | | | |
| CCR5 | ENSG00000160791 | chemokine_(C-C_motif)_receptor_5_{—} | enables | C-C chemokine | 5.695 | 4.992 | 0.000 | 0.000 | 1.653 | 1.262 | 97.062 | 47.311 | 57.487 | 25.312 |
| | | (gene/pseudogene) | | receptor activity | | | | | | | | | | |
| CCR7 | ENSG00000126353 | chemokine_(C-C_motif)_receptor_7 | enables | C-C chemokine | −2.797 | −4.458 | 0.047 | 0.000 | 1.808 | 1.524 | 0.170 | 0.348 | 0.074 | 0.120 |
| | | | | receptor activity | | | | | | | | | | |
| CDC42 | ENSG00000070831 | cell_division_cycle_42 | involved_in | dendritic cell | −2.336 | −2.167 | 0.000 | 0.000 | 4.077 | 0.433 | 0.816 | 0.356 | 0.905 | 0.153 |
| | | | | migration | | | | | | | | | | |
| CDC42EP1 | ENSG00000128283 | CDC42_effector_protein(Rho_{—} | involved_in | Rho protein | −2.653 | −3.206 | 0.000 | 0.000 | 2.593 | 2.171 | 0.417 | 0.129 | 0.287 | 0.174 |
| | | GTPase_binding)_1 | | signal | | | | | | | | | | |
| | | | | transduction | | | | | | | | | | |
| CDC42EP3 | ENSG00000163171 | CDC42_effector_protein(Rho_{—} | involved_in | Rho protein | 0.041 | 0.000 | 0.927 | 0.999 | 9.405 | 4.235 | 9.453 | 1.954 | 9.656 | 1.144 |
| | | GTPase_binding)_3 | | signal | | | | | | | | | | |
| | | | | transduction | | | | | | | | | | |
| CDC42EP4 | ENSG00000179604 | CDC42_effector_protein(Rho_{—} | involved_in | Rho protein | 1.581 | −1.329 | 0.002 | 0.005 | 2.988 | 2.087 | 1.022 | 0.376 | 1.226 | 0.490 |
| | | GTPase_binding)_4 | | signal | | | | | | | | | | |
| | | | | transduction | | | | | | | | | | |
| CFL1 | ENSG00000172757 | cofilin_1_(non-muscle) | involved_in | Rho protein | 1.166 | 0.848 | 0.000 | 0.000 | 95.508 | 12.584 | 214.735 | 48.365 | 172.401 | 45.330 |
| | | | | signal | | | | | | | | | | |
| | | | | transduction | | | | | | | | | | |
| CHUK | ENSG00000213341 | conserved_helix-loop-helix_{—} | involved_in | Rho protein | 0.289 | 0.368 | 0.478 | 0.086 | 6.773 | 1.539 | 8.515 | 3.498 | 8.788 | 0.898 |
| | | ubiquitous_kinase | | signal | | | | | | | | | | |
| | | | | transduction | | | | | | | | | | |
| CIB1 | ENSG00000185043 | calcium_and_integrin_binding_1 | involved_in | thrombopoietin- | −0.845 | −1.098 | 0.002 | 0.000 | 73.725 | 14.932 | 40.644 | 8.448 | 34.433 | 3.129 |
| | | | | mediated | | | | | | | | | | |
| | | | | signaling | | | | | | | | | | |
| | | | | pathway | | | | | | | | | | |
| CNR2 | ENSG00000188822 | cannabinoid_receptor_2 | involved_in | leukocyte | 1.971 | 2.474 | 0.000 | 0.000 | 1.759 | 1.353 | 6.993 | 3.817 | 9.618 | 2.579 |
| | | | | chemotaxis | | | | | | | | | | |
| COL1A2 | ENSG00000164692 | collagen_type_I_alpha_2 | involved_in | Rho protein | −1.300 | −0.392 | 0.001 | 0.327 | 0.435 | 0.187 | 0.182 | 0.037 | 0.343 | 0.130 |
| | | | | signal | | | | | | | | | | |
| | | | | transduction | | | | | | | | | | |
| CORO1A | ENSG00000102879 | coronin_actin_binding_protein_1A | involved_in | leukocyte | 1.236 | 1.026 | 0.000 | 0.000 | 155.760 | 24.417 | 368.945 | 81.425 | 321.428 | 12.606 |
| | | | | chemotaxis | | | | | | | | | | |
| CRK | ENSG00000167193 | v-crk_avian_sarcoma_virus_CT10_{—} | involved_in | helper T cell | 0.444 | 0.247 | 0.157 | 0.364 | 4.438 | 0.967 | 6.189 | 1.615 | 5.400 | 1.406 |
| | | oncogene_homolog | | diapedesis | | | | | | | | | | |
| CRKL | ENSG00000099942 | v-crk_avian_sarcoma_virus_CT10_{—} | involved_in | helper T cell | 0.284 | 0.201 | 0.353 | 0.392 | 26.892 | 7.166 | 33.058 | 5.528 | 31.272 | 3.314 |
| | | oncogene_homolog-like | | diapedesis | | | | | | | | | | |
| CRTAM | ENSG00000109943 | cytotoxic_and_regulatory_T-cell_{—} | involved_in | lymphocyte | −0.149 | 0.761 | 0.858 | 0.215 | 3.015 | 2.231 | 2.807 | 2.639 | 5.134 | 3.917 |
| | | molecule | | migration into | | | | | | | | | | |
| | | | | lymphoid organs | | | | | | | | | | |
| CTNNAL1 | ENSG00000119326 | catenin_alpha-like_1 | involved_in | Rho protein | 3.099 | 3.316 | 0.000 | 0.000 | 0.930 | 0.322 | 8.510 | 3.889 | 9.537 | 1.430 |
| | | | | signal | | | | | | | | | | |
| CX3CR1 | ENSG00000168329 | chemokine(C-X3-C_motif)_receptor_1 | involved_in | chemokine- | 0.931 | 1.152 | 0.113 | 0.041 | 37.022 | 35.395 | 69.040 | 27.179 | 80.391 | 47.748 |
| | | | | mediated | | | | | | | | | | |
| | | | | signaling | | | | | | | | | | |
| | | | | pathway | | | | | | | | | | |
| CXCR2 | ENSG00000180871 | chemokine_(C-X-C_motif)_receptor_2 | enables | chemokine | −3.015 | −2.973 | 0.000 | 0.000 | 5.414 | 2.664 | 0.632 | 0.438 | 0.656 | 0.475 |
| | | | | receptor activity | | | | | | | | | | |
| CXCR3 | ENSG00000186810 | chemokine_(C-X-C_motif)_receptor_3 | enables | C-X-C | 2.261 | 2.149 | 0.000 | 0.000 | 17.999 | 9.538 | 87.328 | 30.989 | 81.097 | 24.460 |
| | | | | chemokine | | | | | | | | | | |
| | | | | receptor activity | | | | | | | | | | |
| CXCR4 | ENSG00000121966 | chemokine(C-X-C_motif)_receptor_4 | enables | C-X-C | −3.456 | −3.277 | 0.000 | 0.000 | 389.521 | 123.58 7 | 35.595 | 12.495 | 40.242 | 20.013 |
| | | | | chemokine | | | | | | | | | | |
| | | | | receptor activity | | | | | | | | | | |
| CXCR6 | ENSG00000172215 | chemokine(C-X-C_motif)_receptor_6 | enables | C-X-C | 5.922 | 5.412 | 0.000 | 0.000 | 0.265 | 0.220 | 17.634 | 14.692 | 12.105 | 11.765 |
| | | | | chemokine | | | | | | | | | | |
| | | | | receptor activity | | | | | | | | | | |
| DNM2 | ENSG00000079805 | dynamin_2 | involved_in | G protein- | 0.154 | −0.029 | 0.604 | 0.862 | 36.192 | 2.948 | 40.284 | 9.935 | 35.873 | 2.238 |
| | | | | coupled receptor | | | | | | | | | | |
| | | | | internalization | | | | | | | | | | |
| DOCK8 | ENSG00000107099 | dedicator_of_cytokinesis_8 | involved_in | dendritic cell | 0.659 | 0.712 | 0.060 | 0.016 | 21.632 | 8.750 | 35.226 | 7.770 | 36.184 | 4.789 |
| | | | | migration | | | | | | | | | | |
| EOGT | ENSG00000163378 | EGF_domain-specific_O-linked_{—} | enables | protein N- | 1.481 | 1.662 | 0.000 | 0.000 | 2.025 | 0.571 | 5.750 | 0.340 | 6.494 | 0.671 |
| | | N-acetylglucosamine_(GlcNAc)_{—} | | acetylglucosaminyl- | | | | | | | | | | |
| | | transferase | | transferase | | | | | | | | | | |
| | | | | activity | | | | | | | | | | |
| FERMT3 | ENSG00000149781 | fermitin_family_member_3 | involved_in | integrin | 0.746 | 0.411 | 0.033 | 0.100 | 77.260 | 22.767 | 130.965 | 41.625 | 105.119 | 6.006 |
| | | | | activation | | | | | | | | | | |
| FFAR2 | ENSG00000126262 | free_fatty_acid_receptor_2 | involved_in | leukocyte | −5.702 | −4.745 | 0.000 | 0.000 | 1.651 | 1.754 | 0.028 | 0.030 | 0.054 | 0.062 |
| | | | | chemotaxis | | | | | | | | | | |
| | | | | involved in | | | | | | | | | | |
| | | | | inflammatory | | | | | | | | | | |
| | | | | response | | | | | | | | | | |
| FUT7 | ENSG00000180549 | fucosyltransferase_7_(alpha_(13)_{—} | involved_in | leukocyte | 2.789 | 2.134 | 0.000 | 0.000 | 2.849 | 0.952 | 19.897 | 7.111 | 12.518 | 5.077 |
| | | fucosyltransferase) | | migration | | | | | | | | | | |
| | | | | involved in | | | | | | | | | | |
| | | | | immune | | | | | | | | | | |
| | | | | response | | | | | | | | | | |
| FUT8 | ENSG00000033170 | fucosyltransferase_8_(alpha_(16)_{—} | enables | alpha-(1->6)- | 1.800 | 1.996 | 0.000 | 0.000 | 1.589 | 0.444 | 5.629 | 2.016 | 6.316 | 1.673 |
| | | fucosyltransferase) | | fucosyl- | | | | | | | | | | |
| | | | | transferase | | | | | | | | | | |
| | | | | activity | | | | | | | | | | |
| GIPR | ENSG00000010310 | gastric_inhibitory_polypeptide_{—} | involved_in | desensitization | −0.972 | −1.115 | 0.052 | 0.004 | 7.498 | 3.919 | 3.757 | 2.193 | 3.528 | 1.490 |
| | | receptor | | of G protein- | | | | | | | | | | |
| | | | | coupled receptor | | | | | | | | | | |
| | | | | signaling | | | | | | | | | | |
| | | | | pathway | | | | | | | | | | |
| GNA12 | ENSG00000146535 | guanine_nucleotide_binding_protein_{—} | involved_in | Rho protein | −1.235 | −1.589 | 0.000 | 0.000 | 14.918 | 5.294 | 6.483 | 1.050 | 5.033 | 1.290 |
| | | (G_protein)_alpha_12 | | signal | | | | | | | | | | |
| | | | | transduction | | | | | | | | | | |
| GNA13 | ENSG00000120063 | guanine_nucleotide_binding_protein_{—} | involved_in | Rho protein | −1.108 | −0.929 | 0.003 | 0.001 | 110.376 | 41.324 | 53.222 | 18.970 | 59.242 | 4.682 |
| | | (G_protein)_alpha_13 | | signal | | | | | | | | | | |
| | | | | transduction | | | | | | | | | | |
| GPR15 | ENSG00000154165 | G_protein-coupled_receptor_15 | involved_in | T cell migration | 7.237 | 7.132 | 0.000 | 0.000 | 0.503 | 0.503 | 83.065 | 39.393 | 74.431 | 25.065 |
| GPR183 | ENSG00000169508 | G_protein-coupled_receptor_183 | involved_in | leukocyte | −8.426 | −8.091 | 0.000 | 0.000 | 71.253 | 36.992 | 0.188 | 0.128 | 0.248 | 0.308 |
| | | | | chemotaxis | | | | | | | | | | |
| GPR75 | ENSG00000119737 | G_protein-coupled_receptor_75 | involved_in | chemokine- | −1.742 | −1.686 | 0.000 | 0.000 | 2.910 | 0.995 | 0.871 | 0.268 | 0.893 | 0.262 |
| | | | | mediated | | | | | | | | | | |
| | | | | signaling | | | | | | | | | | |
| | | | | pathway | | | | | | | | | | |
| GRK4 | ENSG00000125388 | G_protein-coupled_receptor_{—} | involved_in | desensitization | 0.513 | 0.512 | 0.218 | 0.027 | 0.756 | 0.140 | 1.071 | 0.451 | 1.086 | 0.192 |
| | | kinase4 | | of G protein- | | | | | | | | | | |
| | | | | coupled receptor | | | | | | | | | | |
| | | | | signaling | | | | | | | | | | |
| | | | | pathway | | | | | | | | | | |
| HACD3 | ENSG00000074696 | 3-hydroxyacyl-CoA_dehydratase_3 | involved_in | Rho protein | 2.056 | 2.201 | 0.000 | 0.000 | 2.852 | 0.718 | 12.204 | 4.333 | 13.209 | 3.703 |
| | | | | signal | | | | | | | | | | |
| | | | | transduction | | | | | | | | | | |
| ICAM1 | ENSG00000090339 | intercellular_adhesion_{—} | involved_in | T cell | 0.029 | −0.249 | 0.957 | 0.553 | 15.463 | 8.674 | 16.475 | 4.906 | 13.688 | 3.331 |
| | | molecule_1 | | extravasation | | | | | | | | | | |
| IL16 | ENSG00000172349 | interleukin_16 | involved_in | leukocyte | 1.152 | 1.239 | 0.000 | 0.000 | 9.422 | 2.963 | 21.107 | 6.311 | 22.711 | 5.890 |
| | | | | chemotaxis | | | | | | | | | | |
| ITGA1 | ENSG00000213949 | integrin_subunit_alpha_1 | involved_in | cellular | 2.595 | 2.449 | 0.041 | 0.037 | 0.380 | 0.679 | 2.922 | 3.313 | 2.494 | 2.912 |
| | | | | extravasation | | | | | | | | | | |
| ITGA4 | ENSG00000115232 | integrin_subunit_alpha_4 | involved_in | diapedesis | −0.195 | −0.150 | 0.562 | 0.512 | 46.901 | 9.265 | 41.895 | 13.644 | 42.827 | 7.324 |
| ITGA6 | ENSG00000091409 | integrin_subunit_alpha_6 | involved_in | leukocyte | −1.860 | 1.975 | 0.000 | 0.000 | 10.620 | 5.727 | 2.892 | 1.664 | 2.578 | 1.853 |
| | | | | migration | | | | | | | | | | |
| ITGB1BP1 | ENSG00000119185 | integrin_subunit_beta_1_binding_{—} | involved_in | integrin | 1.065 | 0.875 | 0.000 | 0.000 | 6.925 | 1.298 | 14.564 | 2.734 | 12.649 | 3.010 |
| | | protein_1 | | activation | | | | | | | | | | |
| ITGB2 | ENSG00000160255 | integrin_subunit_beta_2 | involved_in | leukocyte | −0.432 | −0.554 | 0.093 | 0.001 | 404.946 | 24.832 | 302.339 | 65.268 | 278.790 | 39.659 |
| | | | | migration | | | | | | | | | | |
| | | | | involved in | | | | | | | | | | |
| | | | | inflammatory | | | | | | | | | | |
| | | | | response | | | | | | | | | | |
| ITGB7 | ENSG00000139626 | integrin_subunit_beta_7 | involved_in | leukocyte | 1.494 | 1.473 | 0.000 | 0.000 | 34.028 | 11.294 | 94.313 | 30.944 | 93.404 | 23.097 |
| | | | | migration | | | | | | | | | | |
| JAM3 | ENSG00000166086 | junctional_adhesion_molecule_3 | involved_in | leukocyte | −0.652 | −0.458 | 0.132 | 0.194 | 0.620 | 0.190 | 0.393 | 0.152 | 0.456 | 0.133 |
| | | | | migration | | | | | | | | | | |
| | | | | involved in | | | | | | | | | | |
| | | | | inflammatory | | | | | | | | | | |
| | | | | response | | | | | | | | | | |
| LIMK1 | ENSG00000106683 | LIM_domain_kinase_1 | involved_in | Rho protein | 0.677 | 0.531 | 0.009 | 0.002 | 18.150 | 1.982 | 28.900 | 7.342 | 26.295 | 4.222 |
| | | | | signal | | | | | | | | | | |
| | | | | transduction | | | | | | | | | | |
| MAPK1 | ENSG00000100030 | mitogen-activated_protein_kinase_1 | enables | MAP kinase | 0.162 | 0.236 | 0.448 | 0.158 | 26.246 | 4.816 | 29.617 | 4.384 | 30.839 | 3.121 |
| | | | | activity | | | | | | | | | | |
| MAPK11 | ENSG00000185386 | mitogen-activated_protein_kinase_11 | enables | MAP kinase | 1.525 | 0.874 | 0.000 | 0.044 | 1.147 | 0.226 | 3.296 | 1.213 | 2.104 | 1.331 |
| | | | | activity | | | | | | | | | | |
| MAPK14 | ENSG00000112062 | mitogen-activated_protein_kinase_14 | enables | MAP kinase | 0.161 | 0.201 | 0.530 | 0.245 | 12.109 | 1.465 | 13.820 | 2.776 | 14.134 | 1.505 |
| | | | | activity | | | | | | | | | | |
| MAPK3 | ENSG00000102882 | mitogen-activated_protein_kinase_3 | enables | MAP kinase | 0.648 | 0.542 | 0.067 | 0.042 | 15.066 | 4.373 | 23.789 | 6.771 | 22.349 | 3.483 |
| | | | | activity | | | | | | | | | | |
| MAPK6 | ENSG00000069956 | mitogen-activated_protein_kinase_6 | enables | MAP kinase | 0.402 | 0.449 | 0.401 | 0.204 | 9.099 | 3.557 | 12.750 | 5.204 | 12.843 | 2.234 |
| | | | | activity | | | | | | | | | | |
| MDK | ENSG00000110492 | midkine_(neurite_growth-promoting_{—} | involved_in | leukocyte | −0.288 | −0.240 | 0.542 | 0.639 | 1.438 | 0.513 | 1.164 | 0.571 | 1.232 | 0.845 |
| | | factor_2) | | chemotaxis | | | | | | | | | | |
| | | | | involved in | | | | | | | | | | |
| | | | | inflammatory | | | | | | | | | | |
| | | | | response | | | | | | | | | | |
| MYO1G | ENSG00000136286 | myosin_IG | involved_in | T cell | 0.654 | 0.415 | 0.030 | 0.049 | 65.666 | 13.715 | 103.997 | 25.364 | 89.067 | 8.944 |
| | | | | meandering | | | | | | | | | | |
| | | | | migration | | | | | | | | | | |
| MYO9B | ENSG00000099331 | myosin_IXB | involved_in | Rho protein | −0.743 | −0.949 | 0.018 | 0.000 | 68.447 | 6.373 | 40.974 | 14.727 | 36.078 | 4.176 |
| | | | | signal | | | | | | | | | | |
| | | | | transduction | | | | | | | | | | |
| NLK | ENSG00000087095 | nemo-like_kinase | enables | MAP kinase | −0.120 | −0.023 | 0.721 | 0.920 | 3.696 | 0.814 | 3.445 | 1.063 | 3.619 | 0.403 |
| | | | | activity | | | | | | | | | | |
| OXSR1 | ENSG00000172939 | oxidative_stress_responsive_1 | involved_in | chemokine (C-C | 0.381 | 0.370 | 0.201 | 0.166 | 12.765 | 1.935 | 17.202 | 4.631 | 16.732 | 3.823 |
| | | | | motif) ligand 21 | | | | | | | | | | |
| | | | | signaling | | | | | | | | | | |
| | | | | pathway | | | | | | | | | | |
| PDE4B | ENSG00000184588 | phosphodiesterase_4B | involved_in | leukocyte | −3.095 | −3.239 | 0.000 | 0.000 | 19.329 | 9.200 | 2.280 | 0.475 | 2.103 | 1.042 |
| | | | | migration | | | | | | | | | | |
| PDE4D | ENSG00000113448 | phosphodiesterase_4D | involved_in | leukocyte | −1.616 | −1.887 | 0.000 | 0.000 | 26.728 | 10.439 | 8.838 | 3.555 | 7.242 | 1.596 |
| | | | | migration | | | | | | | | | | |
| PHACTR4 | ENSG00000204138 | phosphatase_and_actin_regulator_4 | involved_in | Rho protein | −0.175 | −0.064 | 0.446 | 0.786 | 7.548 | 1.088 | 6.729 | 1.131 | 7.222 | 1.475 |
| | | | | signal | | | | | | | | | | |
| | | | | transduction | | | | | | | | | | |
| POFUT2 | ENSG00000186866 | protein_O-fucosyltransferase_2 | enables | fucosyl- | −1.939 | −2.039 | 0.000 | 0.000 | 10.259 | 3.605 | 2.643 | 0.929 | 2.518 | 0.684 |
| | | | | transferase | | | | | | | | | | |
| | | | | activity | | | | | | | | | | |
| PTK2B | ENSG00000120899 | protein_tyrosine_kinase_2_beta | involved_in | chemokine- | −0.341 | −0.457 | 0.189 | 0.000 | 54.364 | 4.230 | 42.888 | 9.517 | 39.956 | 3.269 |
| | | | | mediated | | | | | | | | | | |
| | | | | signaling | | | | | | | | | | |
| | | | | pathway | | | | | | | | | | |
| RAC1 | ENSG00000136238 | ras-related_C3_botulinum_toxin_{—} | involved_in | mast cell | 0.405 | 0.419 | 0.154 | 0.084 | 66.269 | 13.158 | 90.240 | 11.673 | 90.553 | 11.199 |
| | | substrate_1_(rho_family_small_{—} | | chemotaxis | | | | | | | | | | |
| | | GTP_binding_protein_Rac1) | | | | | | | | | | | | |
| RBM15 | ENSG00000162775 | RNA_binding_motif_protein_15 | involved_in | thrombopoietin- | −0.088 | −0.182 | 0.786 | 0.402 | 18.039 | 4.214 | 16.999 | 2.800 | 16.082 | 1.970 |
| | | | | mediated | | | | | | | | | | |
| | | | | signaling | | | | | | | | | | |
| | | | | pathway | | | | | | | | | | |
| RHOA | ENSG00000067560 | ras_homolog_family_member_A | involved_in | Rho protein | 0.369 | 0.464 | 0.147 | 0.015 | 249.151 | 43.125 | 329.276 | 37.978 | 350.389 | 9.143 |
| | | | | signal | | | | | | | | | | |
| | | | | transduction | | | | | | | | | | |
| RHOB | ENSG00000143878 | ras_homolog_family_member_B | involved_in | Rho protein | −2.076 | −1.885 | 0.000 | 0.000 | 25.539 | 11.123 | 5.907 | 3.524 | 6.970 | 3.848 |
| | | | | signal | | | | | | | | | | |
| | | | | transduction | | | | | | | | | | |
| RHOU | ENSG00000116574 | ras_homolog_family_member_U | involved_in | Cdc42 protein | 1.945 | 1.713 | 0.001 | 0.001 | 3.415 | 2.667 | 14.697 | 11.596 | 12.120 | 4.918 |
| | | | | signal | | | | | | | | | | |
| | | | | transduction | | | | | | | | | | |
| ROCK1 | ENSG00000067900 | Rho-associated_coiled-coil_{—} | involved_in | Rho protein | −0.566 | −0.323 | 0.046 | 0.020 | 24.399 | 1.623 | 16.977 | 5.787 | 19.733 | 1.241 |
| | | containing_protein_kinase_1 | | signal | | | | | | | | | | |
| | | | | transduction | | | | | | | | | | |
| ROCK2 | ENSG00000134318 | Rho-associated_coiled-coil_{—} | involved_in | Rho protein | −0.359 | −0.212 | 0.237 | 0.398 | 5.855 | 1.240 | 4.660 | 1.465 | 5.055 | 1.041 |
| | | containing_protein_kinase_2 | | signal | | | | | | | | | | |
| | | | | transduction | | | | | | | | | | |
| RTKN | ENSG00000114993 | rhotekin | involved_in | Rho protein | 1.216 | 0.306 | 0.000 | 0.324 | 1.917 | 0.527 | 4.437 | 1.019 | 2.349 | 0.782 |
| | | | | signal | | | | | | | | | | |
| | | | | transduction | | | | | | | | | | |
| S1PR1 | ENSG00000170989 | sphingosine-1-phosphate_{—} | involved_in | leukocyte | −1.020 | 0.961 | 0.009 | 0.004 | 49.914 | 20.378 | 24.050 | 7.414 | 25.337 | 7.508 |
| | | receptor_1 | | chemotaxis | | | | | | | | | | |
| SH2B3 | ENSG00000111252 | SH2B_adaptor_protein_3 | involved_in | thrombopoietin- | −1.500 | −1.515 | 0.000 | 0.000 | 47.899 | 16.734 | 17.390 | 5.744 | 17.184 | 4.439 |
| | | | | mediated | | | | | | | | | | |
| | | | | signaling | | | | | | | | | | |
| | | | | pathway | | | | | | | | | | |
| STAT5B | ENSG00000173757 | signal_transducer_and_{—} | involved_in | mast cell | −0.170 | −0.137 | 0.450 | 0.403 | 25.944 | 3.516 | 23.199 | 1.319 | 23.816 | 2.252 |
| | | activator_of_transcription_5B | | migration | | | | | | | | | | |
| SYNJ2BP | ENSG00000213463 | synaptojanin_2_binding_protein | involved_in | Rho protein | 0.566 | −0.144 | 0.102 | 0.605 | 2.512 | 0.864 | 1.706 | 0.572 | 2.235 | 0.345 |
| | | | | signal | | | | | | | | | | |
| | | | | transduction | | | | | | | | | | |
| TBX21 | ENSG00000073861 | T-box_21 | involved_in | lymphocyte | 1.410 | 0.989 | 0.000 | 0.000 | 69.557 | 26.590 | 179.645 | 87.673 | 136.785 | 28.677 |
| | | | | migration | | | | | | | | | | |
| TLN1 | ENSG00000137076 | talin_1 | involved_in | integrin | −0.260 | −0.200 | 0.527 | 0.525 | 122.471 | 41.734 | 102.740 | 36.897 | 108.426 | 19.279 |
| | | | | activation | | | | | | | | | | |
| TNF | ENSG00000232810 | tumor_necrosis_factor | involved_in | leukocyte | 4.450 | 3.888 | 0.000 | 0.000 | 3.789 | 1.324 | 86.488 | 29.488 | 59.009 | 27.149 |
| | | | | migration | | | | | | | | | | |
| TRPM2 | ENSG00000142185 | transient_receptor_potential_{—} | involved_in | dendritic cell | −0.363 | −0.108 | 0.463 | 0.817 | 6.990 | 3.588 | 5.412 | 2.585 | 6.552 | 2.738 |
| | | cation_channel_subfamily_{—} | | chemotaxis | | | | | | | | | | |
| | | M_member_2 | | | | | | | | | | | | |
| VAV1 | ENSG00000141968 | vav_guanine_nucleotide_{—} | involved_in | neutrophil | 0.581 | 0.382 | 0.045 | 0.063 | 31.176 | 6.491 | 46.856 | 9.907 | 41.064 | 2.727 |
| | | exchange_factor_1 | | chemotaxis | | | | | | | | | | |
| VAV3 | ENSG00000134215 | vav_guanine_nucleotide_{—} | involved_in | neutrophil | −1.823 | −1.363 | 0.000 | 0.000 | 18.249 | 4.937 | 5.188 | 1.623 | 7.028 | 1.713 |
| | | exchange_factor_3 | | chemotaxis | | | | | | | | | | |

### Example 4

### Chemotactic Receptor Expression Shift on Ex Vivo Expanded NK Cells

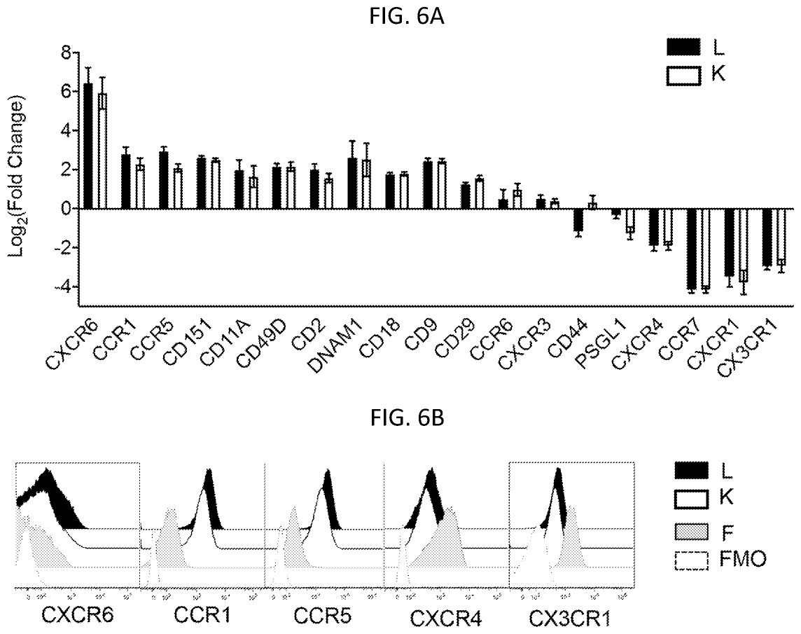

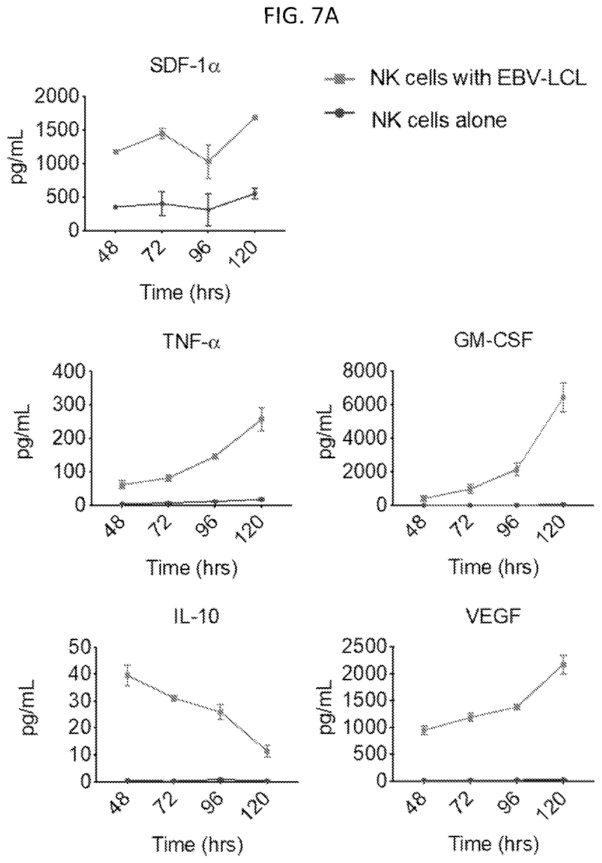

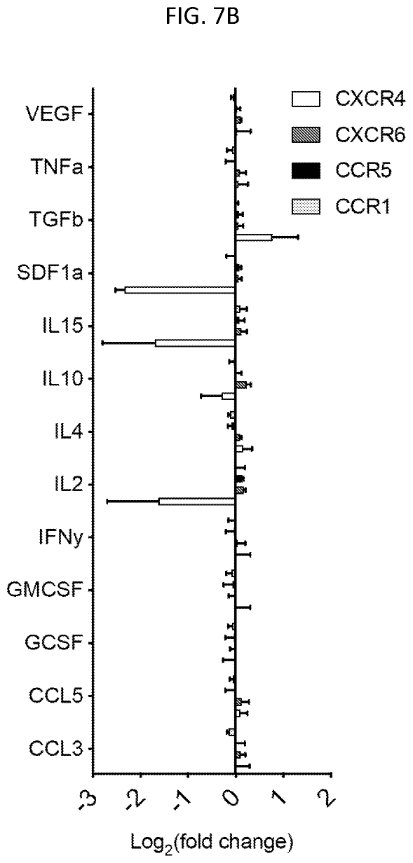

Consistent with previous reports on the effects of ex vivo expansion on NK cell phenotype, the RNA-seq data showing down-regulated CXCR4 transcription corroborated with reduced CXCR4 surface expression observed on expanded NK cells (FIGS. 4A and 4B). As CXCR4 surface expression is critical for cellular trafficking into the bone marrow, these data may account for the low levels of bone marrow homing observed when expanded NK cells were adoptively transferred into rhesus macaques (Sato et al., Clin. Cancer Res. 26:2573-2581, 2020). Surface expression of CCR7, CXCR1, and CX3CR1 all also significantly decreased following ex vivo expansion. In contrast, integrins such as CD11a, b, and c, as well as CD18, CD29, and CD49 all increased on the surface of NK cells (FIG. 6A). Likewise, tetraspanins (plasma membrane organizers that enhance cell adhesion and trafficking) including CD151 and CD9 were also upregulated following expansion. Together, these data suggest that expanded NK cells should have an increased propensity to firmly adhere to tissue endothelium. Compared to the fresh NK cell controls, the chemokine receptors CXCR6, CCR1, CCR5, and CXCR3 increased substantially after expansion. Notably, SMI-EBV-LCL-expanded NK cells showed significantly higher levels of surface CCR1 and CCR5 compared to GE-K562 expanded NK cells (FIGS. 6A and 6B). These chemokine receptors have previously been shown to promote NK cell trafficking from the periphery into the liver during viral infection (Khan et al., PLoS Pathog. 2:e49, 2006; Karlmark et al., Expert Rev. Gastroenterol. Hepatol 2:233-242, 2008). CXCR6, along with CCR5, is involved in retention of liver-resident NK cells within hepatic sinusoids (Stegmann et al., Sci. Rep. 6:26157, 2016; Hudspeth et al., J. Autoimmun. 66:40-50, 2016). Taken together, ex vivo expansion of NK cells with feeder cells and stimulatory cytokines promotes a phenotypic shift, whereby the change in chemokine receptor surface expression might be expected to promote NK cell trafficking to the liver and lessen the capacity for NK cells to travel to the BM (or other tissues). To elucidate the possible mechanisms inducing changes in chemokine receptor phenotype during ex vivo expansion, the role of soluble factors present in culture was investigated. Soluble factors were compared in cultures containing NK cells alone in media with IL-2 media versus NK cell cultures stimulated with irradiated SMI-EBV-LCL feeder cells in the same media. Supernatants collected on days 2, 3, 4, and 5 were analyzed using a multiplex analysis to quantify the presence of GM-CSF, IL-10, IL4, SDF-1α, TNF-α, VEGF, and CCL3, as these factors have previously been identified as possible regulators of chemokine receptor expression on various cell types. In contrast to NK cells in IL-2 media alone, supernatants from NK cells cultured with irradiated EBV-LCL showed increasing concentrations of GM-CSF, TNF-α, and VEGF over time (FIG. 7A). Conversely, the concentration of IL-10, which was increased compared to NK cells stimulated with media, decreased over time, while a sustained elevation was seen with SDF-1α (FIG. 7A).

Next, the role that each of the above soluble factors played in the regulation of chemotactic receptor expression was examined. Fresh NK cells from healthy donor PBMCs were cultured in NK media with various cytokines in vitro for 24 hours. VEGF, TNF-α, IL10, IL4, IFN-γ, GM-CSF, GCSF, CCL5, and CCL3 had no direct effect on the surface expression of CXCR4, CXCR6, CCR5, or CCR1 (FIG. 7B). In contrast, there was a slight increase in CXCR4 expression 24 hours after NK cells were exposed to TGF-β, and a substantial decrease in CXCR4 expression 24 hours after NK cells were exposed to SDF1α, IL-15, and IL-2 (FIG. 7B).

### Example 5

### CRISPR/CAS9 Gene Editing of CCR5 Redirects NK Cell Trafficking In Vivo

Ex vivo-expanded NK cells exhibited a striking upregulation of CCR5, a chemokine receptor associated with trafficking to and retention in the liver. Therefore, CRISPR gene editing of this chemokine receptor was tested as a strategy to redirect NK cell trafficking in vivo in immunodeficient mice receiving infusions of ex vivo expanded human NK cells.

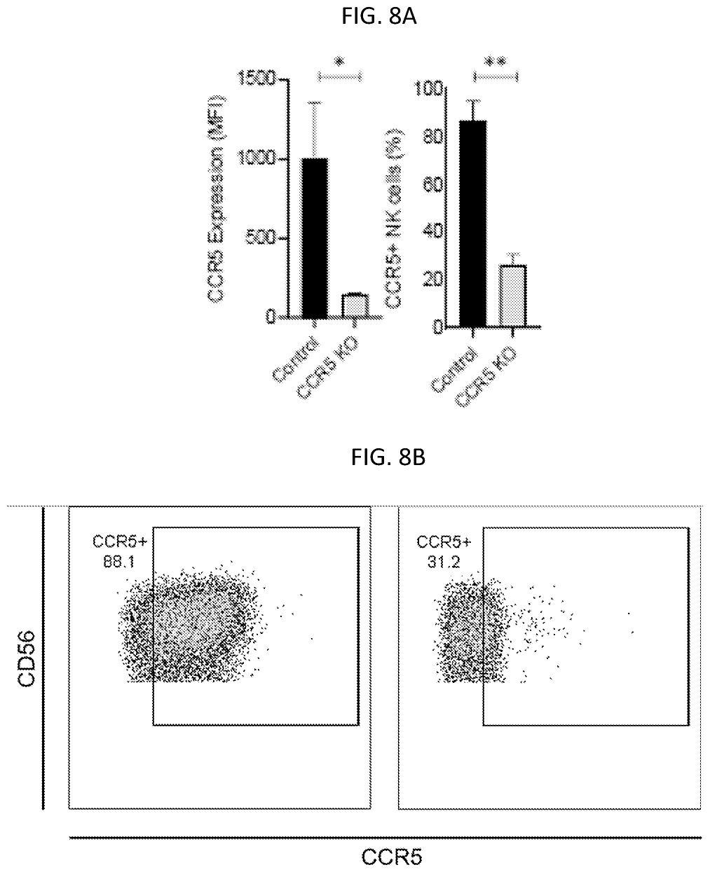

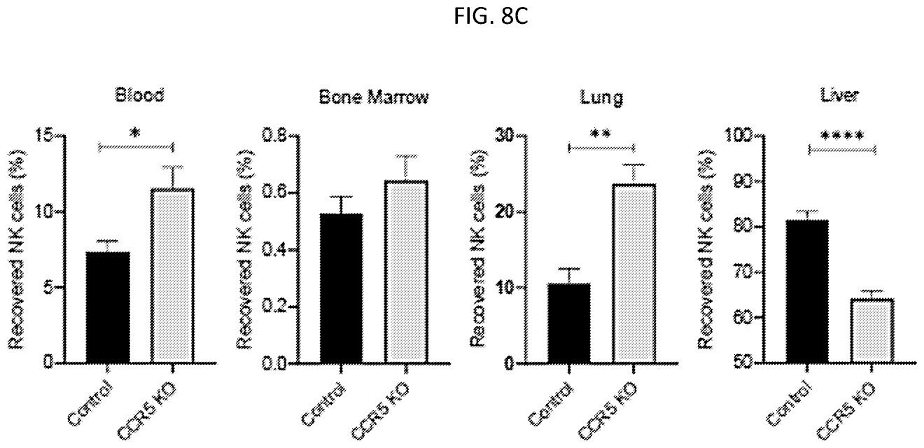

Seven days following ex vivo expansion, NK cells were electroporated with a mix of 3 sgRNAs (SEQ ID NOs: 1-3) targeting CCR5 complexed with Cas9. Electroporated cultures harvested on day 14 maintained their proliferative capacity and had substantially reduced CCR5 expression (22%) compared to non-electroporated control NK cells (88%; FIGS. 8A and 8B). Both expanded NK cell populations were then injected i.v. into NSG mice, with blood, BM, lungs, and livers being harvested 24 hours following infusion. Disrupting CCR5 in NK cells significantly reduced cellular trafficking into the liver compared to control NK cells (FIG. 8C). Moreover, there were significantly more CCR5 CRISPR/Cas9 disrupted NK cells in the circulation and a significantly higher percentage of these cells in the circulation and in the lung compared to control NK cells. These data suggest that the upregulation CCR5 in expanded NK cells may promote NK cell trafficking from the circulation to the liver tissue following i.v. infusion.

### Example 6

### Methods of Treating a Subject with Cancer

This example describes methods of treating a subject with cancer with modified NK cells with reduced CCR5 expression. An exemplary protocol is illustrated schematically in FIG. 9. However, one skilled in the art will appreciate that methods that deviate from these specific methods can also be used to successfully treat or inhibit cancer in a subject.

A subject with cancer, for example multiple myeloma, undergoes apheresis to collect peripheral blood mononuclear cells. NK cells (e.g., CD56-positive/CD3-negative cells) are isolated from the PBMCs by positive and/or negative selection using immuno-magnetic methods. The isolated NK cells are activated and/or expanded ex vivo, for example by culture on feeder cells (such as SMI-EBV-LCL or K562.mbIL-21.41BBL feeder cells) in the presence of 500 U/mL IL-2 for about 3-5 days. On about day 6, the expanded NK cells are electroporated with a mix of sgRNAs targeting CCR5 complexed with Cas9. Other gene modifications can also be made at the same time, such as CRISPR/Cas9 editing of CCXCR6, CCR1, and/or CD38 or transduction with a nucleic acid encoding CXCR4 or other chemokine receptors for specific tissue targeting. The NK cells can also be transduced with a nucleic acid encoding a chimeric antigen receptor specific for the cancer being treated, for instance a CD38-CAR for multiple myeloma.

The modified NK cells are maintained in expansion culture for about 3-15 days. On about day 8-12, for example, on day 10, the expanded modified NK cells can be cryopreserved for later use or formulated for administration to the subject (for example, in a pharmaceutically acceptable carrier). A composition comprising 10⁶ to 10¹² of the expanded NK cells is administered to the subject intravenously. Patients with multiple myeloma expressing CD38 may also be treated with an anti-CD38 antibody such as daratumumab. The response of the subject's cancer is monitored periodically, for example, up to 1 year, up to 3 years, up to 5 years, or more.

In view of the many possible embodiments to which the principles of the disclosure may be applied, it should be recognized that illustrated embodiments are only examples and should not be considered a limitation on the scope of the invention. Rather, the scope of the invention is defined by the following claims. We therefore claim as our invention all that comes within the scope and spirit of these claims.

1. A modified ex vivo expanded natural killer (NK) cell comprising reduced expression of C-C chemokine receptor 5 (CCR5) compared to an unmodified ex vivo expanded NK cell.

2. The modified NK cell of claim 1, comprising a deletion of at least one nucleotide and/or an insertion of at least one nucleotide in genomic DNA encoding CCR5.

3. The modified NK cell of claim 2, wherein the deletion and/or insertion is in exon 2 of CCR5.

4. The modified NK cell of claim 1, wherein the cell comprises a reduced amount of mRNA encoding CCR5 compared to an unmodified ex vivo expanded NK cells.

5. The modified NK cell of claim 1, further comprising a heterologous nucleic acid encoding C-X-C chemokine receptor 4 (CXCR4), a heterologous nucleic acid encoding C-C chemokine receptor 7 (CCR7), a heterologous nucleic acid encoding C-X-C chemokine receptor 3 (CXCR3), or a combination of two or more thereof.

6. The modified NK cell of claim 5, wherein the cell comprises increased expression of CXCR4, CCR7, or CXCR3 compared to an unmodified ex vivo expanded NK cell.

7. The modified NK cell of claim 1, further comprising reduced expression of C-C chemokine receptor 1 (CCR1), C-X-C chemokine receptor 6 (CXCR6), CD38, or a combination of two or more thereof compared to an unmodified ex vivo expanded NK cell.

8. The modified NK cell of claim 1, further comprising a chimeric antigen receptor.

9. A composition comprising the modified NK cell of claim 1 and a pharmaceutically acceptable carrier.

10. A method of treating a subject with cancer, comprising administering to the subject an effective amount of the modified NK cell of claim 1.

11. The method of claim 10, wherein the modified NK cells are autologous to the subject with cancer.

12. The method of claim 10, wherein the subject with cancer has a solid tumor or a hematological malignancy.

13. (canceled)

14. The method of claim 10, further comprising administering to the subject an anti-cancer antibody.

15. A method of producing the modified NK cell of claim 1, comprising:
expanding a population of isolated NK cells in a cell culture medium comprising interleukin-2, interleukin-15, or both for 2-5 days to produce a population of expanded NK cells;
introducing a ribonucleoprotein comprising at least one sgRNA targeting CCR5 and Cas9 into the population of expanded NK cells to produce a population of modified NK cells; and
culturing the population of modified NK cells in a cell culture medium comprising interleukin-2, interleukin-15, or both for 3-15 days to produce the modified NK cells.

16. The method of claim 15, wherein expanding the population of NK cells and/or culturing the population of modified NK cells comprises culturing the NK cells in the presence of irradiated feeder cells.

17. The method of claim 16, wherein the irradiated feeder cells comprise an Epstein-Barr virus transformed lymphoblastoid cell line or a genetically modified K562 cell line.

18. The method of claim 15, wherein the cell culture medium comprises 500 IU/ml IL-2 and/or wherein the cell culture medium comprises 10 mg/ml IL-15.

19. (canceled)

20. The method of claim 15, wherein the at least one sgRNA has the nucleic acid sequence of any one of SEQ ID NOs: 1-3.

21. The method of claim 15, further comprising introducing into the expanded NK cells a ribonucleoprotein comprising at least one sgRNA targeting CCR1, at least one sgRNA targeting CXCR6, and/or at least one sgRNA targeting CD38, and Cas9 and/or further comprising transducing the expanded NK cells with a viral vector comprising one or more heterologous nucleic acids encoding one or more of CXCR3, CXCR4, CCR7, and a chimeric antigen receptor.

22-23. (canceled)

24. The method of claim 15, further comprising formulating the modified NK cells with a pharmaceutically acceptable carrier.


SEQUENCE LISTING (3 sequences)

SEQ ID NO: 1 - RNA, 20 nt, Artificial Sequence
  Synthetic guide RNA
uuuugcaguuuaucaggaug

SEQ ID NO: 2 - RNA, 20 nt, Artificial Sequence
  Synthetic guide RNA
aaaacaggucagagauggcc

SEQ ID NO: 3 - RNA, 20 nt, Artificial Sequence
  Synthetic guide RNA
uguauuuccaaagucccacu